(19) United States
(12) Patent Application Publication
(10) Pub. No.: US 2005/0154410 A1
(43) Pub. Date: Jul. 14, 2005

(54) Lancing device and multi-lancet cartridge

(76) Inventors: William E. Conway, Smyrna, GA (US); Christopher J. Ruf, Marietta, GA (US); John C. Irwin, Woodstock, GA (US); Stephen J. Flynn, Peachtree City, GA (US); Avi M. Robbins, Longwood, FL (US); Brian D. VanHiel, Smyrna, GA (US); Brian D. Leutz, McDonough, GA (US); Richard W. LeVaughn, Newnan, GA (US); Michael V. Lipoma, Villa Rica, GA (US)

Correspondence Address: GARDNER GROFF, P.C., 2018 POWERS FERRY ROAD, SUITE 800, ATLANTA, GA, 30339, US

(21) Appl. No.: 10/987,591

(22) Filed: Nov. 12, 2004

Related U.S. Application Data
(60) Provisional application No. 60/519,232, filed on Nov. 12, 2003.

(51) Int. Cl. A61B 17/32

(52) U.S. Cl. 606/181

(57) ABSTRACT

A lancing device and a removable and replaceable multi-lancet cartridge for use in connection therewith are disclosed. The multi-lancet cartridge is advanced through sequential lancets, and anti-reverse features prevent reverse advancement of the cartridge. Interlocks are provided to prevent double-cocking the device, to reduce the likelihood of jamming of the mechanism. Improved trigger mechanisms and depth control mechanisms are provided.

### CROSS REFERENCE TO RELATED APPLICATIONS

This application claims the benefit of U.S. Provisional Patent Application Ser. No. 60/519,232, filed Nov. 12, 2003; the content of which is hereby incorporated herein by reference in its entirety.

### TECHNICAL FIELD

The present invention relates generally to medical devices and procedures, and to related methods of manufacture; and more particularly to lancing devices for the collection and/or analysis of samples of blood or other bodily fluids, and to replaceable multi-lancet cartridge assemblies for use in connection with such lancing devices.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics and in blood typing and screening applications.

In some instances, a person must periodically sample their blood for multiple testing throughout the day or week. Because re-use of a lancet can result in infection or spread of blood borne contaminants, persons requiring repeated testing often must carry multiple lancets with them, which are separately loaded into a lancing device for each sampling. This can be inconvenient and may lead to reduced compliance with a prescribed test regimen. Additionally, the need for repeated loading and unloading of lancets into a lancing device can be quite conspicuous and distracting to others when done in public, resulting in reduced compliance by some users.

Accordingly, improved lancing devices have been developed, which are capable of carrying out multiple sampling procedures without the need for separately loading individual lancets. For example, PCT International Publication No. WO 03/071940 A1 (International Application No. PCT/US03/05159, filed 20 Feb. 2003), which is incorporated herein by reference, discloses a lancing device including a replaceable multi-lancet cartridge. Such lancing devices provide considerable advantage over single-lancet devices, warranting continued development of related technologies capable of providing further improved convenience and discretion in use.

It is to the provision of an improved sampling device and cartridge meeting these and other needs that the present invention is primarily directed.

### SUMMARY OF THE INVENTION

Briefly described, example embodiments of the present invention include an improved sampling device that is convenient, compact, and includes multiple lancets in a single replaceable cassette or cartridge. The improvements of the present invention preferably increase convenience and discretion for the user, thereby encouraging more frequent testing and insuring compliance with the subject's prescribed testing regimen.

In one aspect, the present invention is a lancing device including a plurality of lancets; an advancing mechanism for sequentially advancing the lancets in a first direction and bringing an active one of the lancets into engagement with a drive mechanism; and an anti-reverse mechanism for preventing advancement of the lancets in a second direction opposite the first direction.

In another aspect, the invention is a lancing device including a replaceable multi-lancet cartridge, a drive mechanism for driving an active lancet of the multi-lancet cartridge between a cocked position and a lancing position, and an interlock for preventing advancement of the cartridge when the drive mechanism is cocked.

In still another aspect, the invention is a lancing device including a plurality of lancets in an array, and an advancing mechanism to advance through the array of lancets and successively bring each of the plurality of lancets into an active position. The advancing mechanism preferably includes indexing means to prevent partial advancement of the array to a position where no lancet is in the active position.

In another aspect, the invention is a lancing device including a replaceable multi-lancet cartridge having a plurality of lancets associated therewith, and lancet retaining means to prevent the lancets from being displaced from the cartridge until advanced into an active position.

In another aspect, the invention is a replaceable multi-lancet cartridge for a lancing device, the cartridge including a plurality of lancets, each lancet having a removable endcap associated therewith, and at least one spring for biasing removed endcaps out of a path of travel of their associated lancets, the at least one spring being initially flat, and being flexed upon assembly into the cartridge.

In yet another aspect, the invention is a lancing device including a drive piston, a drive spring for advancing the drive piston, and a return spring for retracting the drive piston. One of the drive spring or the return spring surrounds an exterior portion of the drive piston, and the other spring is captured within an internal recess of the drive piston.

In another aspect, the invention is a lancing device including a drive piston movable between a cocked position and a fired position. The lancing device further includes a trigger mechanism having a release member defining an aperture, wherein an expanded portion of the drive piston is releasably engageable within the aperture of the release member to retain the drive piston in its cocked position until released.

In another aspect, the invention is a lancing device for use in combination with a multi-lancet cartridge comprising a plurality of lancets, the lancing device including a housing, a drive mechanism, an advancing mechanism for bringing the lancets of the multi-lancet cartridge sequentially into engagement with the drive mechanism, and a depth-control mechanism providing adjustment of a depth of penetration of an active one of the lancets.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is a perspective schematic view of a lancing device and a cartridge according to an example embodiment of the invention

FIGS. 2a and 2b are a perspective and an assembly view of a multi-lancet cartridge according to an example embodiment of the invention.

FIG. 3 is a perspective view of a lancing device according to another example embodiment of the invention, having a cartridge loaded therein, and with an upper portion of the housing removed for clarity.

FIG. 4 shows the drive mechanism portion of a lancing device according to another example embodiment of the invention, including a ratchet mechanism for preventing partial cocking.

FIGS. 5a-5c show a torsion spring mechanism for advancing through sequential lancets of a lancet cartridge, and to prevent reverse movement and/or partial advancement between lancets, in a lancing device according to another example embodiment of the invention.

FIG. 6 shows a rotating advancer mechanism of a lancing device for advancing through sequential lancets of a lancet cartridge, according to an example embodiment of the invention.

FIGS. 7a and 7b show a leaf spring mechanism for indexing and advancement through sequential lancets of a lancet cartridge, and to prevent reverse movement of the cartridge, according to another example embodiment of the invention.

FIGS. 8a and 8b show an indexer arm mechanism for engagement with cooperating features of a lancet cartridge to index and advance through sequential lancets of a lancet cartridge, and to prevent reverse movement of the cartridge, according to another example embodiment of the invention.

FIG. 9 shows an embodiment of a lancet cartridge having a lancet-retaining ring for preventing lancets from moving radially until advanced into a firing position.

FIGS. 10a and 10b show an embodiment of a lancet cartridge having a plurality of molded cantilevers in the cartridge top cover for lancet retention.

FIGS. 11a and 11b show a cartridge embodiment having a flat stamped spring ring with a plurality of individual spring loops for biasing removed lancet caps out of the lancet's travel path.

FIG. 12 shows a cartridge embodiment having transversely-biased spring members for biasing removed lancet caps out of the lancet's travel path.

FIGS. 13a-13c show different embodiments of spring clip members for retraction of the protective endcap of each individual lancet.

FIG. 14 shows a cartridge embodiment having lancet hold-down snaps for retaining individual lancets.

FIG. 15 shows an anti-rotation interlock mechanism for locking the cartridge when the drive mechanism is cocked and allowing advancement of the cartridge after firing, according to an example embodiment of the invention.

FIGS. 16a and 16b show an anti-rotation interlock mechanism according to another embodiment of the invention.

FIG. 17 shows another embodiment of a lancing device according to the present invention, having a spring-biased plunger for indexing and advancement through sequential lancets of a lancet cartridge, and to prevent reverse movement of the cartridge.

FIG. 18 shows another embodiment of a lancing device, including a cantilevered spring arm for preventing double-cocking of the lancing device.

FIG. 19 shows a lancet drive mechanism of a lancing device according to an example embodiment of the invention, having an in-line configuration of the drive and return springs.

FIG. 20 shows a lancet drive mechanism of a lancing device according to another embodiment of the invention, having the drive and return springs in a laterally-offset configuration.

FIG. 21 shows a trigger mechanism for a lancing device according to an example embodiment of the invention, having a transversely-sliding cage for engaging and releasing the drive piston.

FIGS. 22-26 show trigger mechanisms according to alternate embodiments of the invention, having shutter mechanisms for engaging and releasing the drive piston.

FIGS. 27a and 27b show another embodiment of a trigger mechanism for a lancing device, having a flexing trigger button arm with a shutter aperture at its free end.

FIG. 28 shows another embodiment of a trigger mechanism for a lancing device, having a flexing trigger button element integrally molded with the drive piston.

FIGS. 29a and 29b show another embodiment of a trigger mechanism for a lancing device, having a hinged trigger button.

FIG. 30 shows a rotational depth-control mechanism for a lancing device, according to an example embodiment of the invention.

FIG. 31 shows a pivotal depth-control member for a lancing device.

FIG. 32 shows a translational depth-control mechanism for a lancing device.

FIG. 33 shows a screw-driven translational depth-control member for a lancing device.

FIGS. 34-36 show several alternate embodiments of a rotating disk depth-control member for a lancing device.

FIG. 37 shows a cam slot driven translational depth-control mechanism for a lancing device.

FIG. 38 shows a screw slot driven translational depth-control mechanism for a lancing device.

FIG. 39 shows a geared translational depth-control mechanism for a lancing device.

FIG. 40 shows a multi-shutter depth-control mechanism for a lancing device.

FIG. 41 shows alternate forms of direct and indirect actuation of depth-control mechanisms for a lancing device.

FIGS. 42 and 43 show a alternate forms of pivotal depth-control mechanisms for a lancing device.

FIG. 44 shows an extensible iris depth-control mechanism for a lancing device.

FIG. 45 shows a pivoting panel depth-control mechanism for a lancing device.

FIG. 46 shows a slotted depth-control mechanism for a lancing device.

FIG. 47 shows a flexible strip depth-control mechanism for a lancing device.

FIG. 48 shows a gear-driven translational depth-control mechanism for a lancing device.

FIG. 49 shows a removable member depth-control mechanism for a lancing device.

FIGS. 50-53 show alternate embodiments of rotational member depth-control mechanisms for a lancing device.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

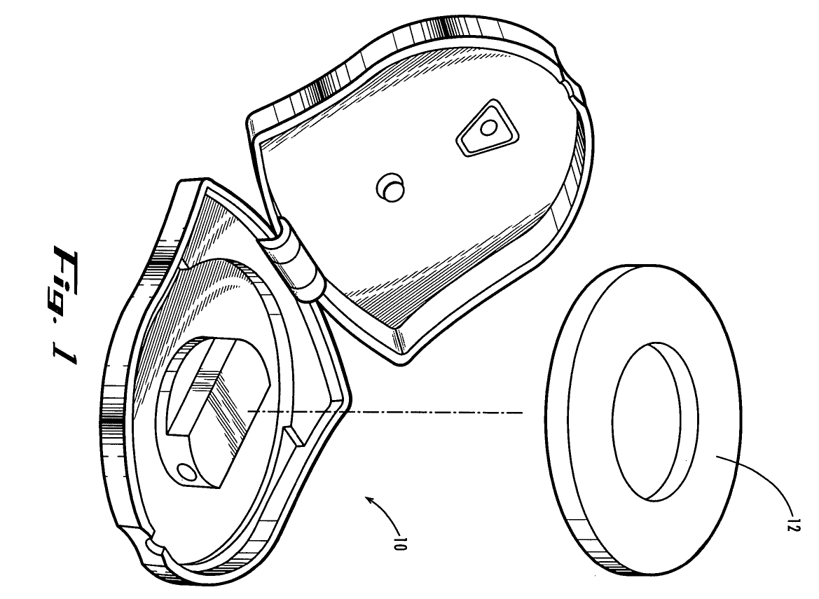

In its various embodiments, the present invention provides an improved lancing device 10, preferably for use in combination with a replaceable multi-lancet cartridge 12, as shown schematically for example in FIG. 1. While the improvements of the present invention are adaptable for application in connection with various forms of multi-lancet lancing devices, PCT International Publication No. WO 03/071940 A1, incorporated herein by reference, shows an example form of a multi-lancet lancing device to which the improvements of the present invention are of potential application. It will be recognized that the improvements disclosed herein are of individual advantage, or can be used in combination with one another. In general, the lancing device 10 of the present invention comprises a housing defining a chamber for receiving the cartridge; a drive mechanism for propelling an active lancet of the cartridge through a lancing stroke, from a retracted position within the cartridge to an advanced position wherein a sharp tip of the active lancet projects through a lancet opening in the housing to pierce the subject's skin at an intended lancing site; a charging mechanism for energizing the drive mechanism; and an advancing mechanism for sequentially advancing lancets of the cartridge into and through the active position. Various of these mechanisms can be combined; for example, a single mechanism optionally serves to energize the drive mechanism and simultaneously or sequentially advance the cartridge.

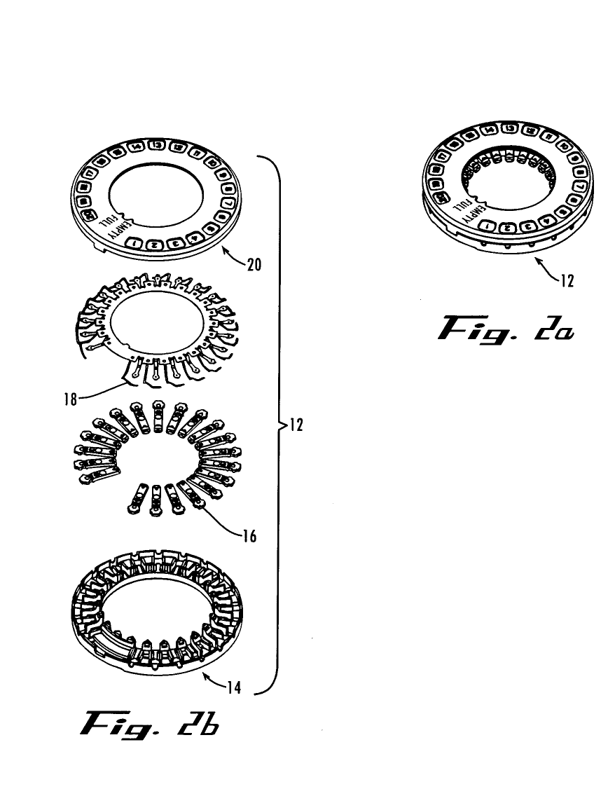

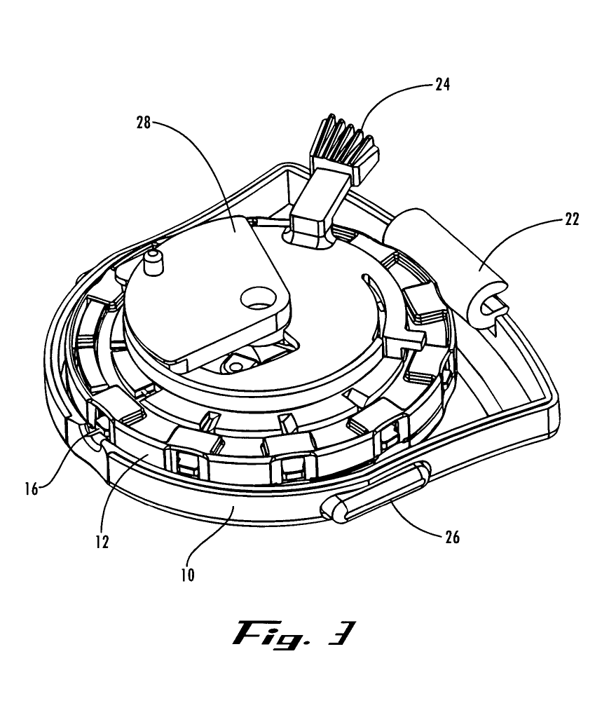

FIG. 2 shows a replaceable multi-lancet cartridge 12 according to an example embodiment of the present invention. The cartridge 12 preferably comprises a lancet carrier or base 14, preferably defining a plurality of lancet guide tracks through which individual lancets traverse upon actuation, defining their respective lancing strokes. The cartridge 12 preferably comprises a plurality of lancets 16, each slideable within a corresponding guide track. Each lancet 16 preferably comprises a lancet body having a sharp lancet tip projecting therefrom, and a removable protective endcap covering the sharp lancet tip. One or more biasing members 18 are preferably provided, for moving protective endcaps removed from the lancets out of the path of travel of each active lancet as it traverses its lancing stroke. A cover 20 preferably overlies the lancets and couples with the base 14 to provide an enclosure. The cover optionally comprises numerical or other indicia, observable from the exterior of the lancing device when in use, showing which lancet is in use, and/or how many lancets remain for use, and/or that the device is full (unused cartridge) and/or empty (fully used cartridge).

FIG. 3 shows a lancing device 10, with its upper housing half-shell removed for clarity, having a multi-lancet cartridge 12 installed therein. The upper housing half-shell would be pivotally connected to the lower housing half-shell by a hinge coupling 22, as partially depicted. An arm 24 is pivotally operable to advance the cartridge to bring sequential lancets 16 of the cartridge into the active position, and to energize the drive mechanism and de-cap the active lancet. An activation button or trigger 26 releases the drive mechanism to propel the active lancet through its lancing stroke. A depth-control mechanism 28 is provided, to allow the user to selectively adjust the penetration depth of the lancet.

Advancing and Indexing

Example embodiments of the lancing device of the present invention preferably include one or more mechanisms for advancing through sequential lancets of a multi-lancet cartridge or lancet array, for indexing the advancement to prevent partial advancement of the cartridge to a position between lancets wherein no lancet is in the active position, to prevent double-cocking the device and thereby advancing a lancet into and through the active position without using that lancet, and/or to prevent reverse operation and potential re-use of a lancet.

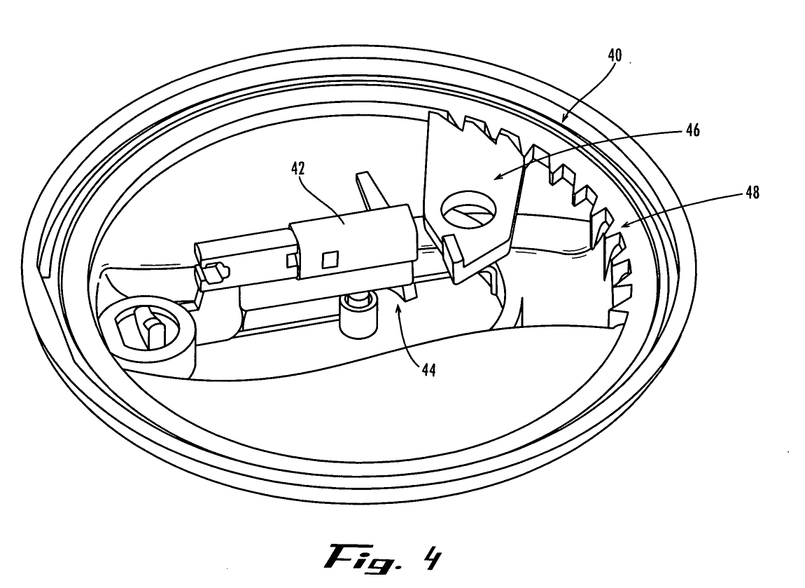

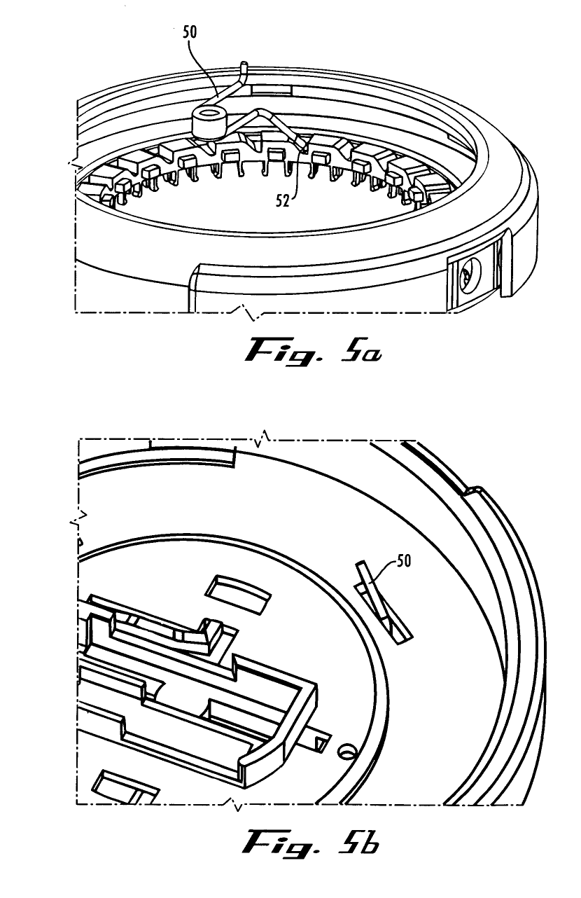

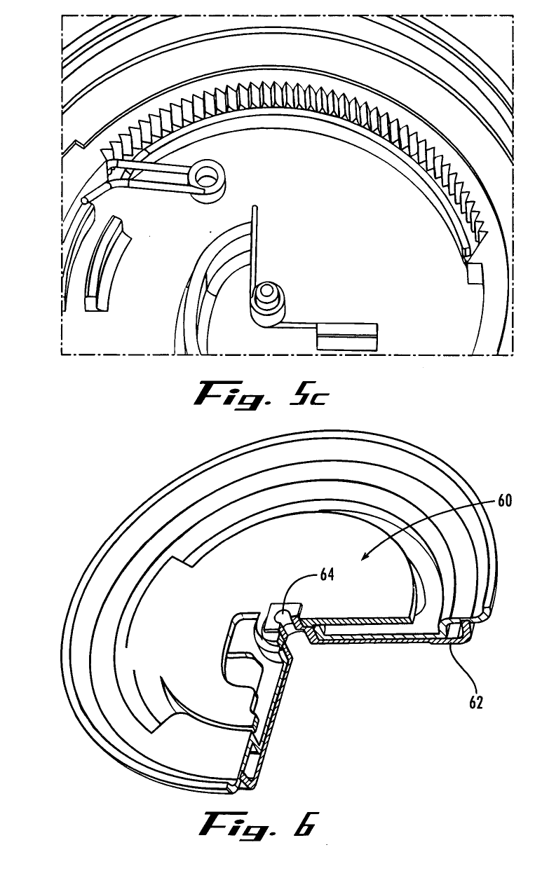

For example, FIG. 4 shows an anti-reverse mechanism for a multi-lancet lancing device according to an example embodiment of the present invention. A ratchet and pawl mechanism 40 allows the device to be advanced through sequential lancets of a lancet array in or on a lancet cartridge, and prevents reverse movement and/or partial advancement between lancets. As the drive piston 42 is energized, a contact surface 44 preferably engages the pawl 46 at the limit of its travel, releasing the ratchet 48 to allow the advancing and/or arming mechanism to return to its normal or default position. In this manner, the advancing mechanism operates in a back-and-forth manner, wherein the user actuates the mechanism in a first or forward direction in engagement with the cartridge to advance the cartridge to the next lancet, and in a second or reverse direction out of engagement with the cartridge to return the mechanism to its original postion. In alternate embodiments, the advancing mechanism is actuated continuously in one direction (i.e., clockwise or counter-clockwise) through all the lancets of the cartridge, without the need for any return stroke.

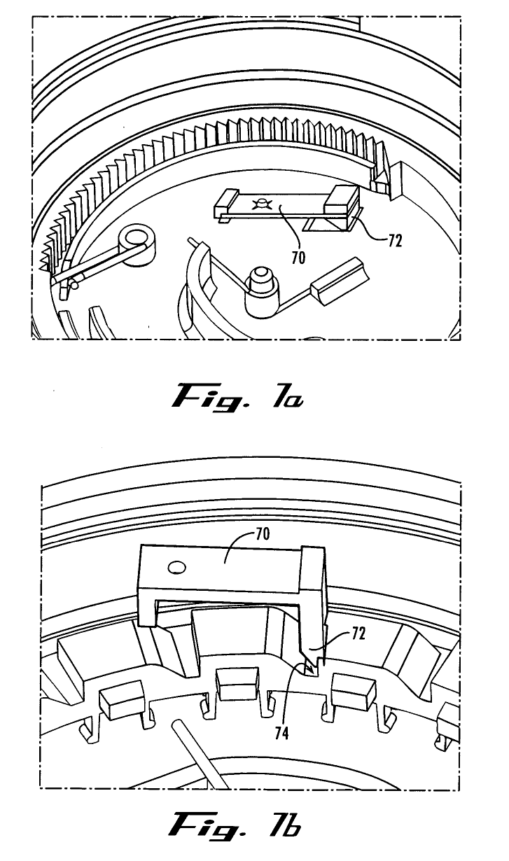

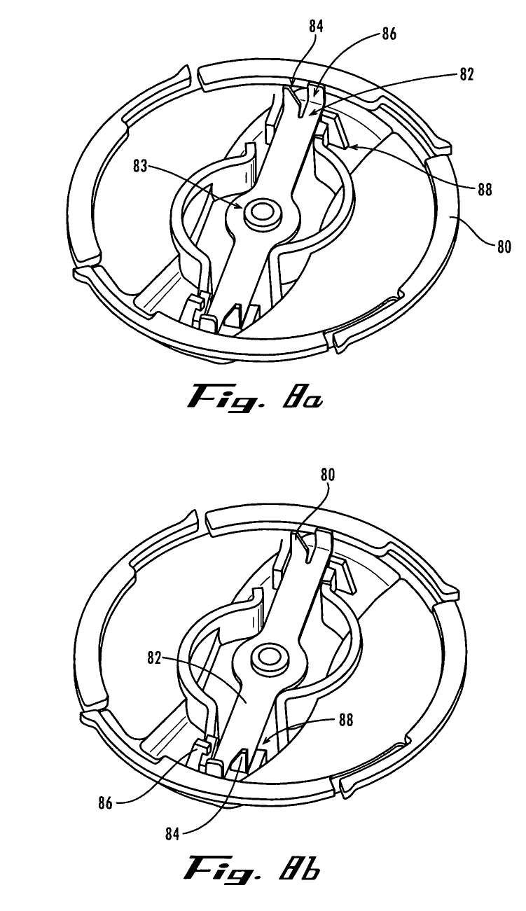

FIGS. 5a-5c show another embodiment of an anti-reverse mechanism, including a torsion spring 50 for engagement with cooperating detents 52 in the cartridge, forming a ratchet mechanism to allow the cartridge to advance through sequential lancets, and to prevent reverse movement of the cartridge. The mechanism preferably also provides indexing of the advancement through sequential lancets, to prevent partial advancement between lancets. In alternate embodiments, a frictional clutch mechanism is provided to prevent reverse movement of the cartridge.

FIG. 6 shows a rotational advancing mechanism 60 for a multi-lancet lancing device according to an example embodiment of the invention. The advancing mechanism includes an external rotating advancer knob 62, coupled to an internal advancer 64 that engages the cartridge to advance through sequential lancets when the user rotationally actuates the knob.

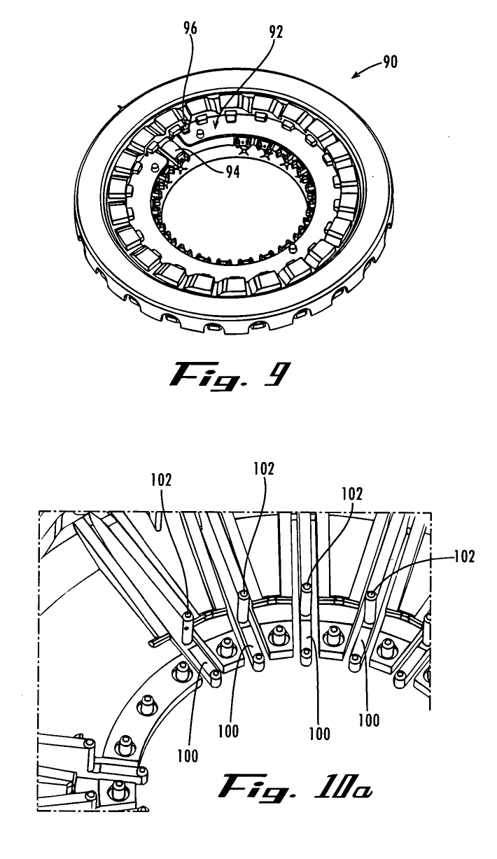

FIGS. 7a and 7b show an anti-reverse mechanism for a multi-lancet lancing device according to an example embodiment of the invention, including a leaf spring 70 having a tooth or finger 72 for engagement with cooperating detents 74 in the lancet cartridge for indexing and advancement through sequential lancets of the cartridge, and to prevent reverse movement of the cartridge.

FIGS. 8a and 8b show an advancing knob 80 having an indexer arm 82 mounted, as by heat staking or adhesive, to a central hub 83 of the knob. The arm 82 is preferably formed as a double-ended flexible metal member. Each end of the arm preferably includes a first angled finger 84 for engagement with cooperating features of the lancet cartridge to index and advance the cartridge through sequential lancets, and a second angled finger 86 for tracking a cam surface within the lancing device housing to flex the indexer arm 82 into and out of engagement with the cartridge at the appropriate location of the actuation sequence. This mechanism allows the advancing knob 80 to be rotationally actuated in a single direction, 180° per index step, without the need for a return stroke. Symmetrical guidance and advancing features 88 are preferably arranged adjacent the angled fingers at either end.

Lancet and Endcap Retention

Example embodiments of the lancing device of the present invention preferably also comprise features for retaining lancets in position in or on a multi-lancet cartridge until advanced into engagement with the drive mechanism at the active lancet position. In this manner, inadvertent discharge of lancets from a cartridge, jamming, and/or noise due to rattling of loose lancets in a cartridge is prevented. The lancing device preferably also includes features for displacing protective endcaps that are removed from the active lancet out of the lancet's path of travel along the lancing stroke, and retaining the removed endcaps.

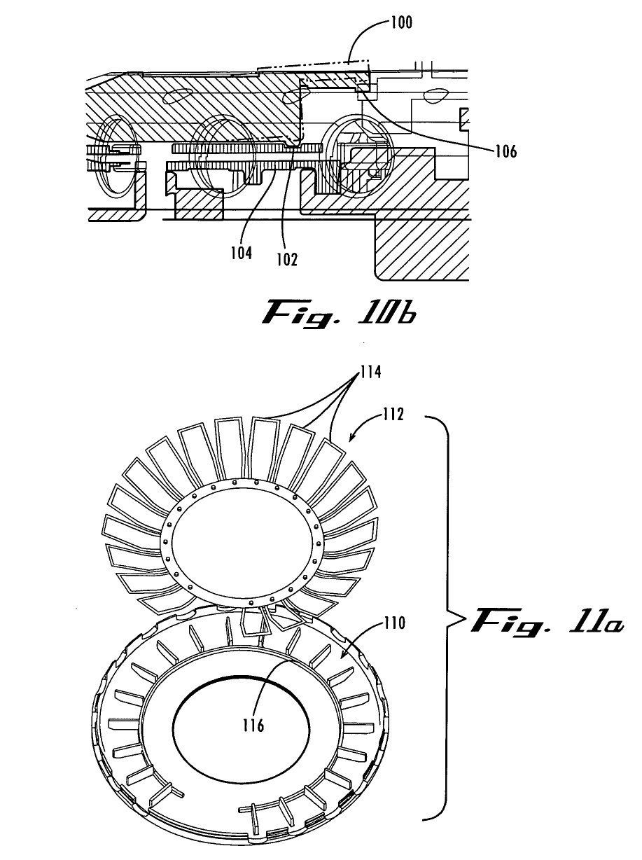

For example, FIG. 9 shows a multi-lancet cartridge 90 according to an example embodiment of the invention having a lancet-retaining ring 92 for preventing lancets from moving radially until advanced into a firing or “active” position beneath a split or open segment of the ring 92. A retaining pin or projection 94 can be provided on each lancet for engagement with a cooperating track, channel or edge of the ring. The ring 92 optionally includes a limit member 96 to prevent further advancement of the lancet cartridge after it has been advanced through all of the lancets, to prevent re-use of lancets.

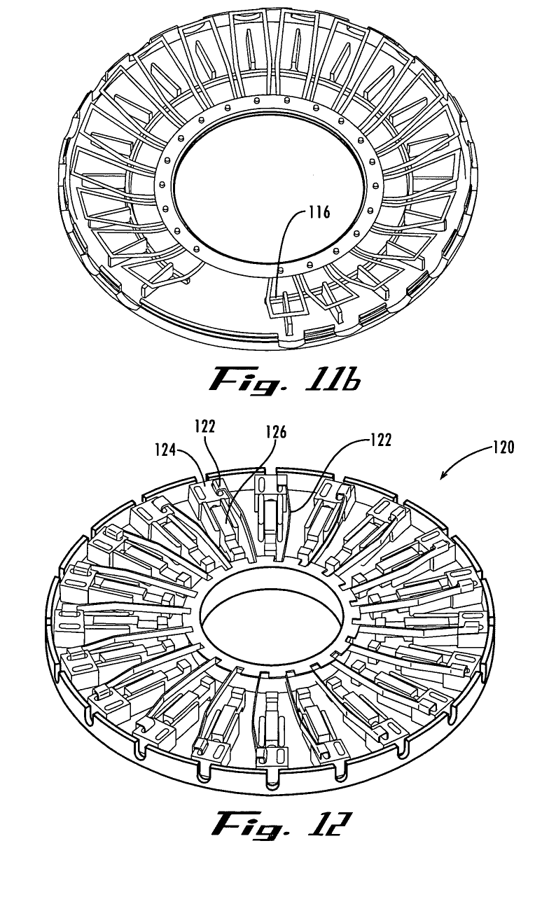

FIGS. 10a and 10b show an embodiment of the invention having a plurality of molded cantilevers 100 formed in a top cover portion of the cartridge for retaining lancets in position in the cartridge until the lancet is advanced into the active position. A lancet retaining pin 102 on each cantilever engages a respective lancet 104 to secure it in position, until the lancet is advanced into the active position, wherein a lifter 106 engages the cantilever 100 to flex it out of engagement with the active lancet 104, releasing the lancet for firing.

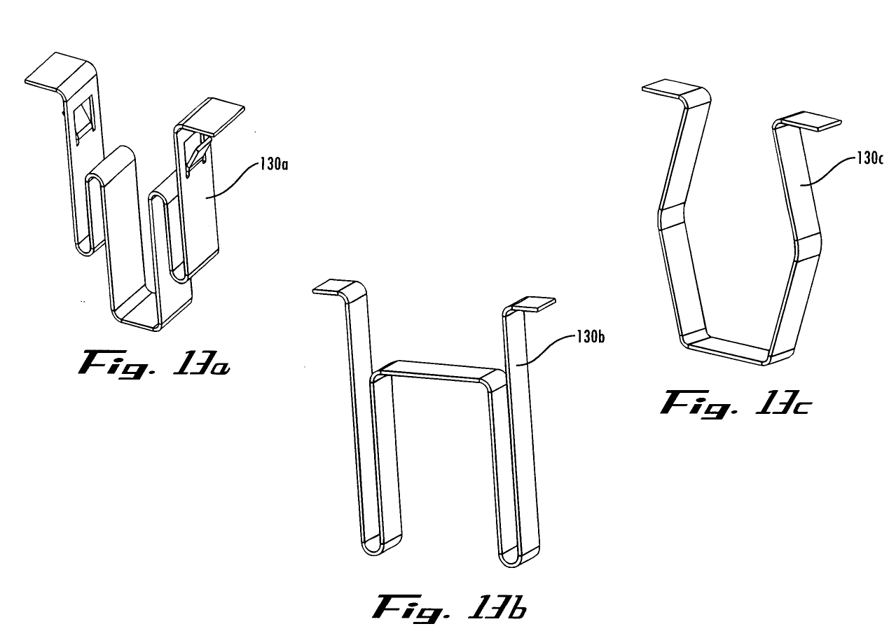

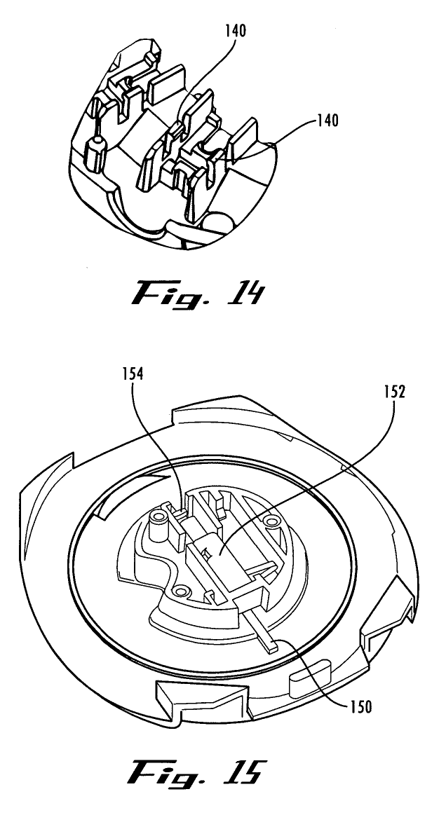

FIGS. 11a and 11b show a cartridge cover 110 having a flat stamped spring ring 112 with a plurality of individual spring loops 114 mounted thereon. The spring ring 112 is preferably attached to the cover 110 by press-fitting holes of the spring ring onto Tinnerman-style pins, and a raised ring or flange 116 projecting from the cover causes each of spring loops 114 to be outwardly deflected during assembly to preload the loops to bias the endcaps out of the plane of the lancet array upon removal from the lancet at the active position. The provision of a flat spring ring mounted in this manner has been found to provide easier and less expensive manufacture and handling during assembly than forming a pre-bent spring member. For further advantage during assembly, a plurality of such spring rings may be provided in a connected strip, or in a stacked array, for automated dispensing during production.

FIG. 12 shows a cartridge 120 having a transversely-biased spring member 122 engaging the protective cap 124 of each lancet 126, for lateral, in-plane removal of the lancet cap. The spring member 122 is pre-loaded upon assembly, so that upon removal of the cap 124 from a lancet 126, the spring member pulls the cap to the side, out of the active lancet's path of travel. This configuration allows removed endcaps to be stored in the same plane as the lancet array, yet still out of the active lancet's path of travel, thereby reducing the necessary cartridge thickness as compared to cartridges that store the removed endcaps in a well or recess beneath or above the plane of the lancets.

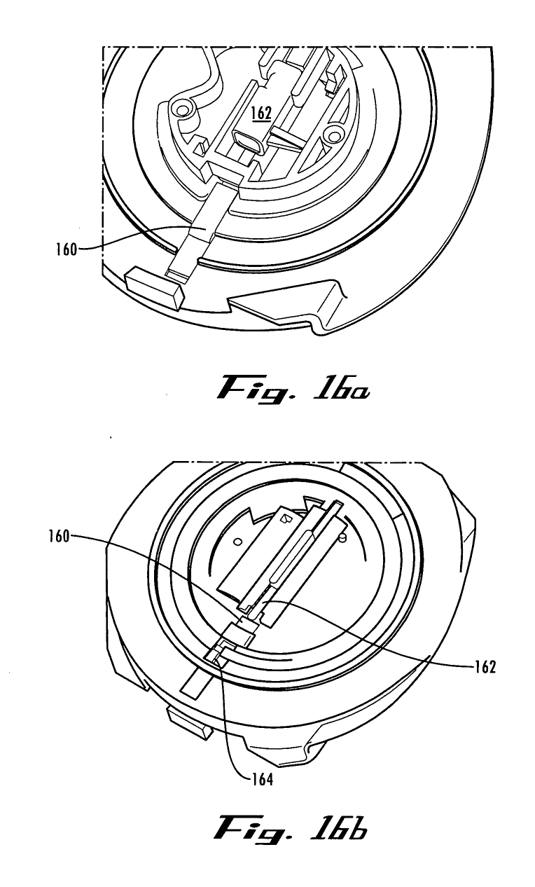

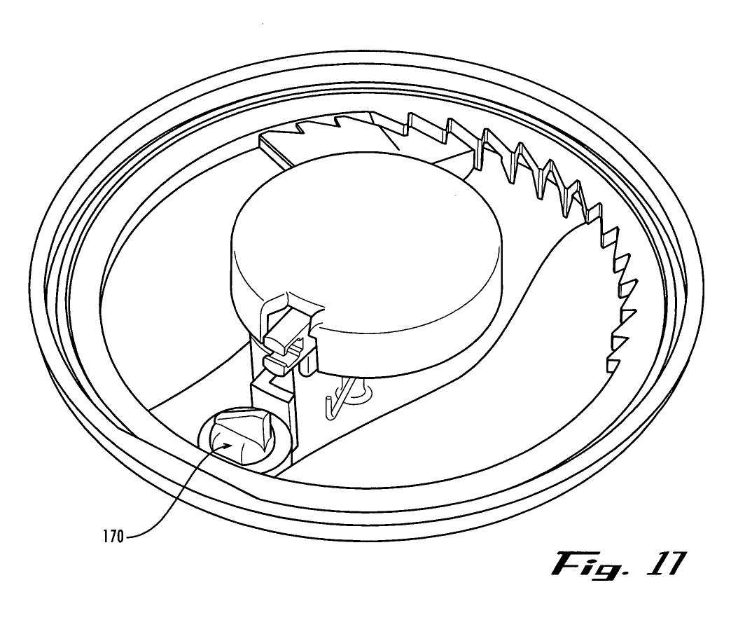

FIGS. 13a-13c show alternate embodiments of individual spring clips 130, for use in a multi-lancet cartridge providing a separate spring clip member for engagement and retraction of the protective endcap of each individual lancet. In further alternate embodiments, two or more (for example, 2, 4, 5 or 10) connected spring clips are formed as a spring segment or strip, and multiple segments are installed with a spring clip engaging each lancet endcap.

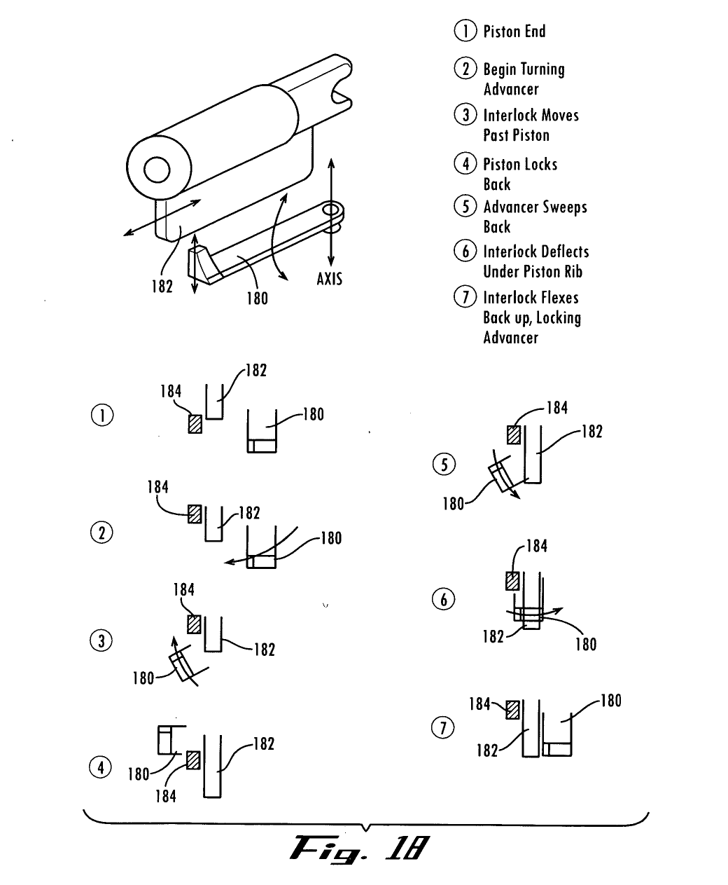

FIG. 14 shows a portion of a cartridge according to another embodiment of the invention, having resilient lancet hold-down snaps 140 for holding the lancets in place during assembly and use. The lancet is able to slide freely between the hold-down snaps, but the snaps prevent the lancets from being displaced from the cartridge. The snaps optionally also serve as directional guides for the lancet's travel during firing.

Cartridge Alignment

Example embodiments of the lancing device of the present invention preferably also include anti-rotation interlock features for fixing the cartridge in position when the drive mechanism of the device is charged (i.e., in its cocked configuration). In this manner, proper lancet alignment is maintained, and vibration and play in the drive mechanism are reduced.

For example, FIG. 15 shows an anti-rotation interlock mechanism for a multi-lancet lancing device, comprising an arm 150 projecting from the distal end of the drive piston 152, opposite the lancet engaging jaw 154. The arm engages a cooperating recess or other surface feature of the lancet cartridge when the drive mechanism is cocked, to prevent motion of the cartridge, but to allow advancement of the cartridge after firing.

In other example embodiments, such as shown in FIGS. 16a and 16b, the anti-rotation interlock features comprise a locking bolt 160, which is retracted upon contact with the drive piston 162 when the drive mechanism is cocked, to engage the lancet cartridge and lock it in position. A projection 164 on the lower face of the locking bolt 160 interfaces with a cooperating feature on the advancing mechanism to block further advancement of the cartridge and thereby prevent double-cocking and/or potential jamming. In other embodiments, the device includes a ratcheting retaining ring for retaining the lancets in place and for preventing double-cocking and/or re-use by preventing rotation of the lancet cartridge when the device is cocked.

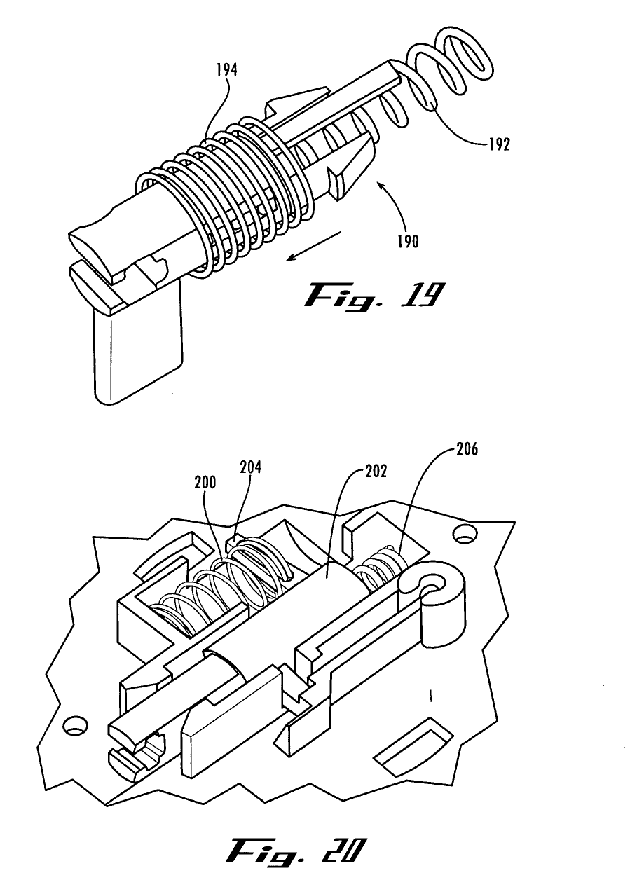

FIG. 17 shows an embodiment of the invention including a spring-biased plunger 170 having an inclined shoulder for engagement with cooperating detents in the lancet cartridge to index the cartridge as it is advanced through sequential lancets of the cartridge, and to prevent reverse movement of the cartridge.

The multi-lancet cartridge of the present invention optionally also includes a break-away section that differentiates a new cartridge from a used cartridge, to prevent accidental re-use of a potentially contaminated lancet. For example, a flag or indicator can be provided for manual displacement by the user, or which is automatically broken off of the cartridge or otherwise displaced upon insertion into the housing or upon initial advancement or firing. In its various embodiments, the cartridge can be assembled using assembly methods including one or more of: ultra-sonic welding, snaps, crush pins, solvent bonding, adhesive, thermal welding, and/or laser welding.

Drive Mechanism and Actuation

Example embodiments of the lancing device of the present invention preferably also include an improved drive mechanism, and/or an improved actuation (i.e., trigger) mechanism. For example, FIG. 18 shows a portion of a drive mechanism for a multi-lancet lancing device according to an example embodiment of the invention, including a cantilevered flexing interlock spring arm 180 for engagement with a cooperating interlock fin 182 on the drive piston, and a re-inforcement rib 184 in the mechanism base, to prevent double-cocking of the lancing device, thereby reducing the likelihood of jamming. In the depicted sequence of operation, the piston 182 is in its forward (fired) position when the user begins turning the advancer. The interlock arm 180 moves past the piston, and the piston locks in its rearward (charged) position. The advancer is then moved back along its return stroke, and the interlock arm 180 deflects under the piston 182. The interlock 180 then flexes back up, locking the advancer. The piston is charged, and the reinforcement rib 184 prevents the interlock fin of the piston 182 from bending.

FIG. 19 shows a drive mechanism including an in-line piston assembly 190, having one of the drive spring 192 or return spring 194 externally mounted thereon, abutting against an exterior shoulder; and the other of the drive or return springs internally mounted within a bore in the distal end of the piston, in a coaxial nested manner. The opposed springs operate in tandem to advance and retract the piston (and the active lancet coupled thereto) through its lancing stroke.

FIG. 20 shows another embodiment of a drive mechanism, including a laterally offset retraction spring 200 alongside the drive piston 202, and in the plane of the array of lancets in or on the cartridge. The retraction spring 200 operates against a laterally projecting arm 204 of the piston 202. The drive spring 206 is in line with the piston 202, and operates against the distal end of the piston.

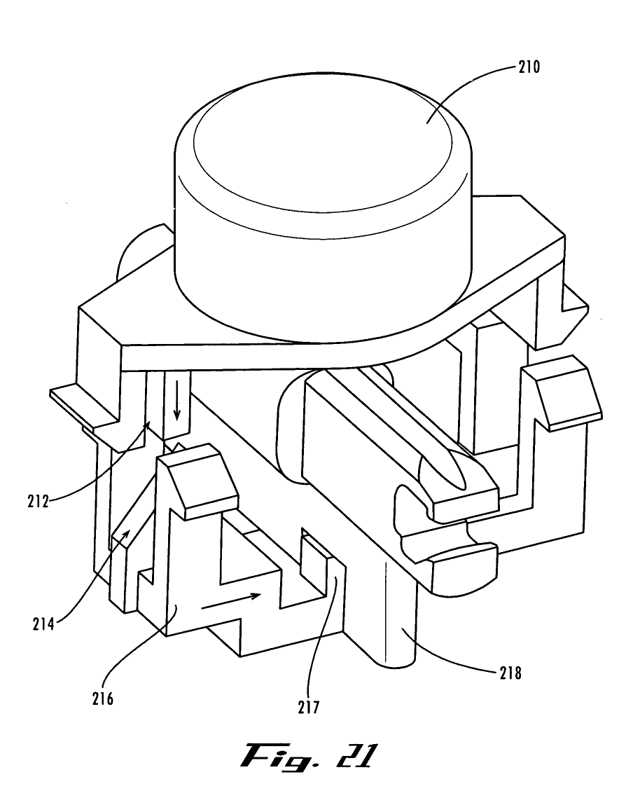

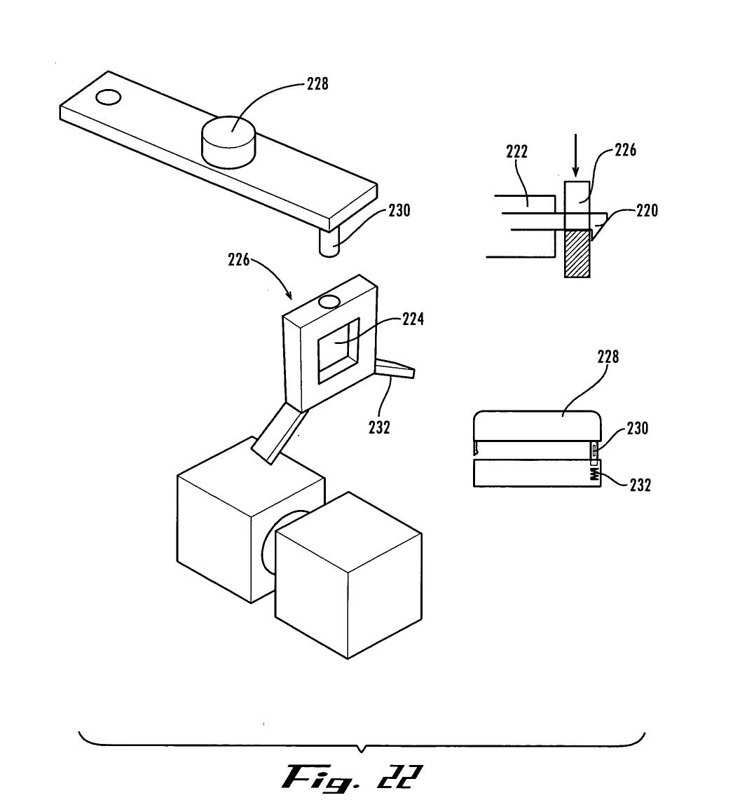

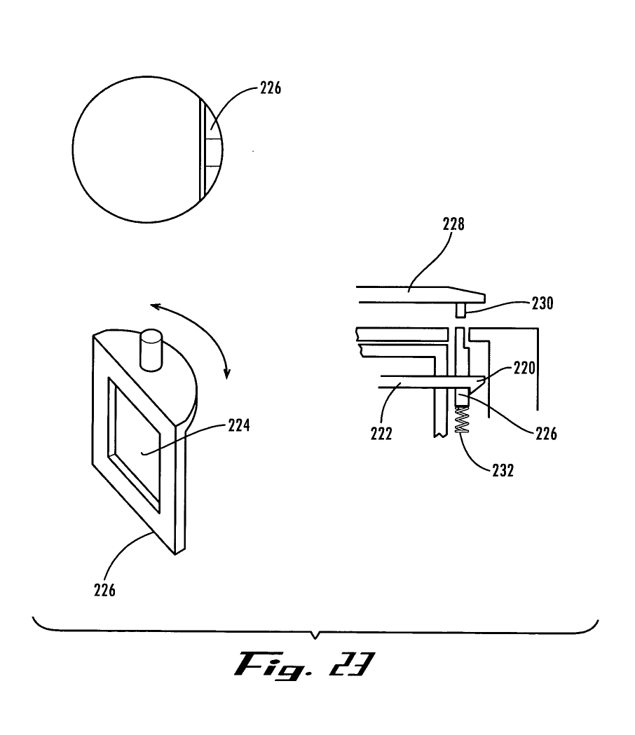

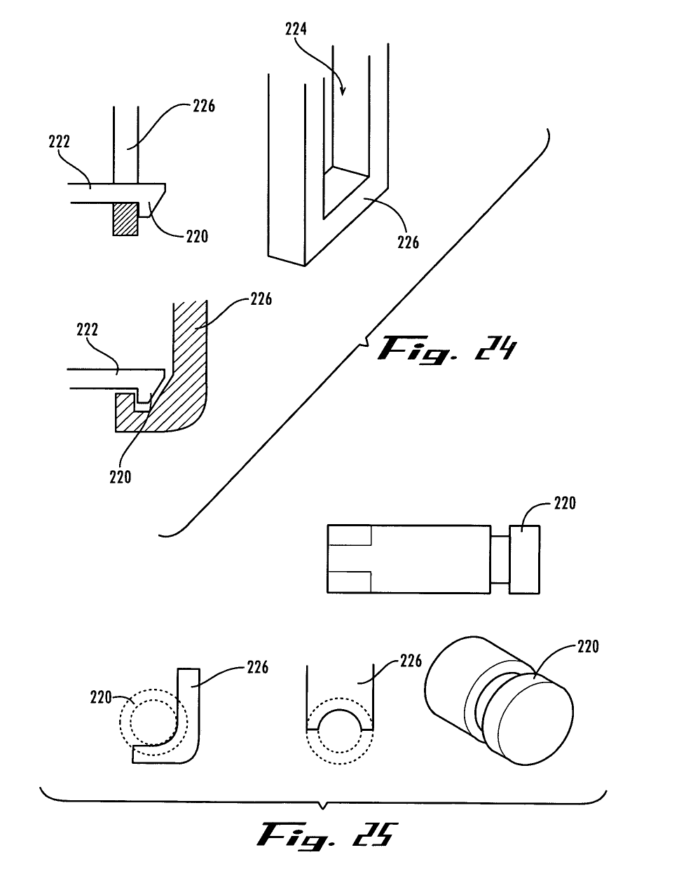

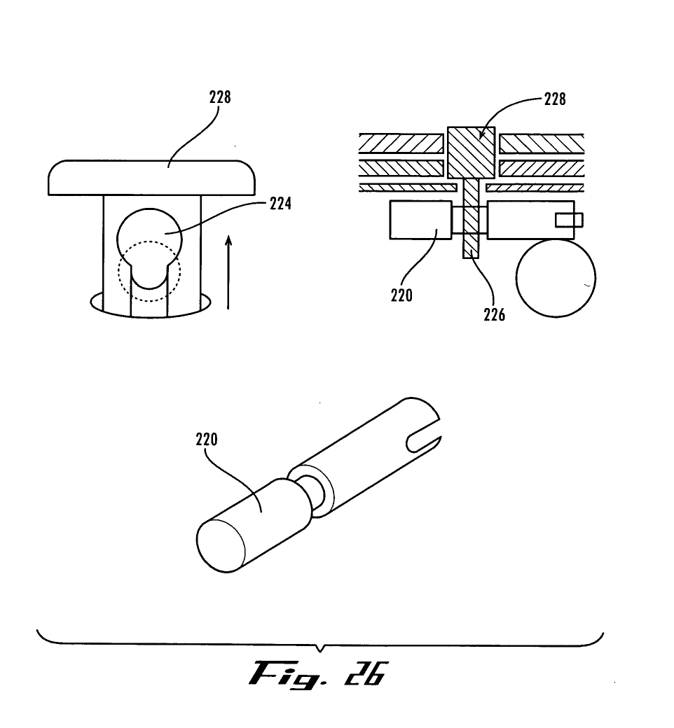

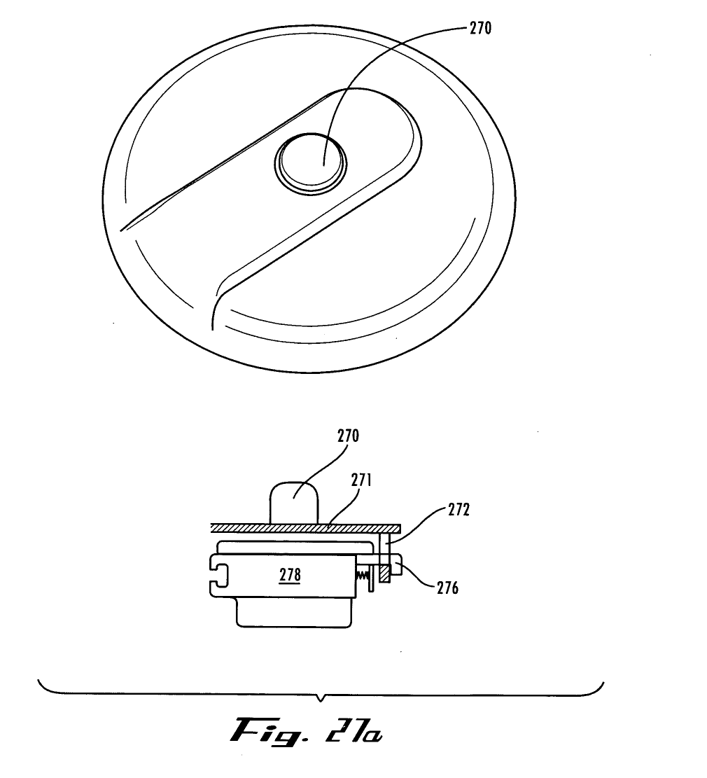

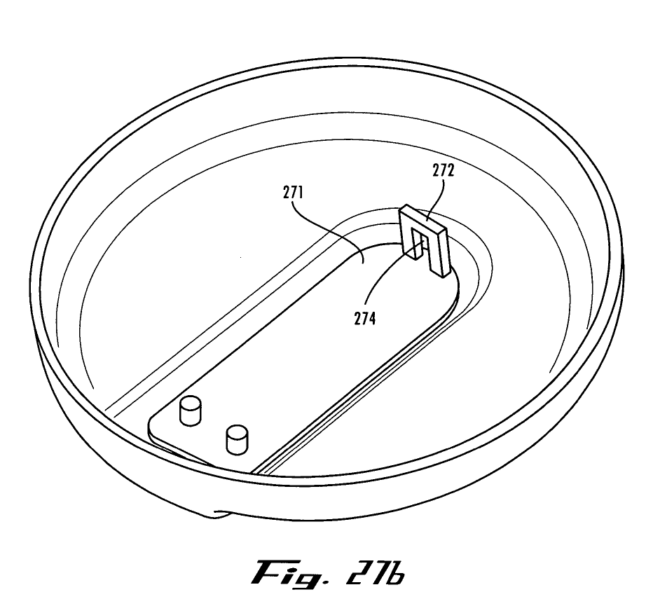

FIG. 21 shows an improved trigger mechanism according to an example embodiment of the invention. Actuation of the release button 210 drives a finger 212 connected to the button along an inclined surface 214 of a sliding cage 216, moving the cage transversely, and moving a sear surface 217 out of engagement with the drive piston 218 to release the piston and fire the device.

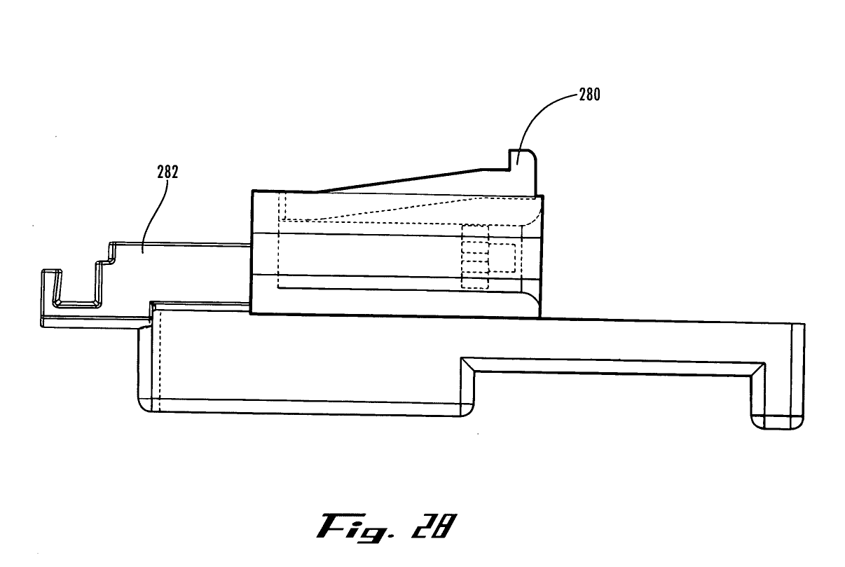

FIGS. 22-26 show various alternate embodiments of shutter trigger mechanisms according to example forms of the invention. In general, a barb or expanded portion 220 extending from the drive piston 222 releasably engages within an aperture 224 formed in a shutter or release member 226. The release member 226 is actuated to release the barb or expanded portion 220 from engagement with the aperture 224 to fire the lancing device. In the embodiment of FIG. 26, a keyhole aperture 224 includes a large diameter portion allowing passage of expanded portion 220 therethrough, and a smaller diameter portion for engagement with a portion of the drive piston having a reduced diameter. An actuator button 228 or other member is preferably provided external of the lancing device housing, with a finger or other projection 230 extending through the housing into contact with the release member to actuate the device. One or more spring members 232 are preferably provided to return the shutter to the ready state after firing.

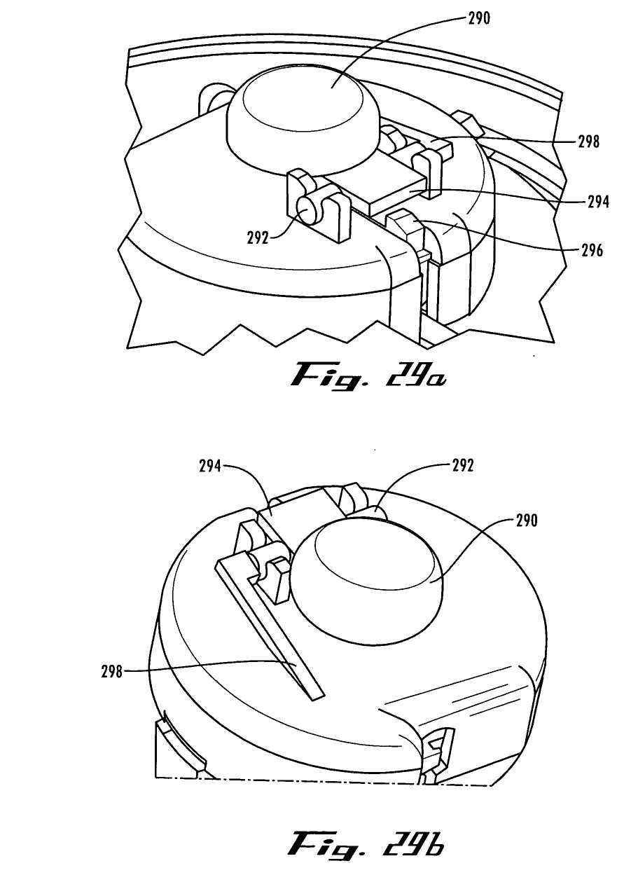

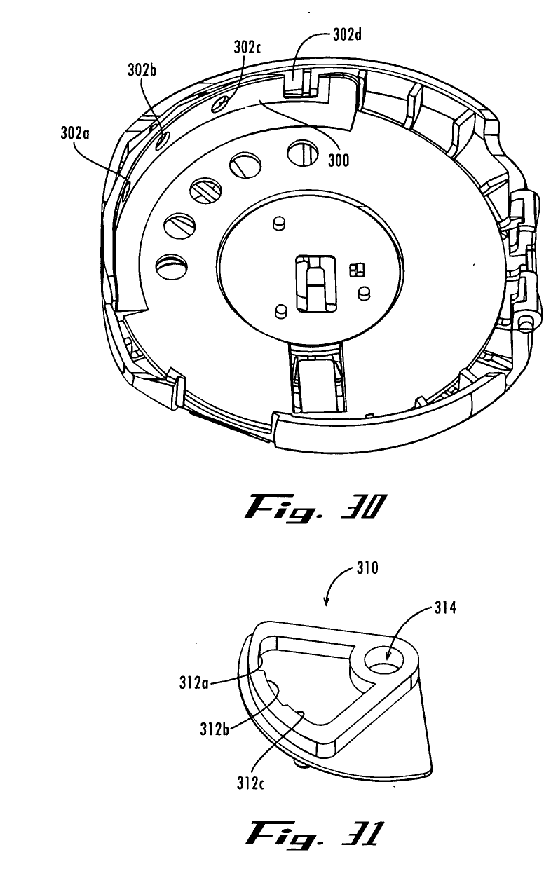

FIGS. 27a and 27b show a flexing trigger button mechanism having an actuator button 270 mounted to a flexing arm 271, coupled to a release member 272 having an aperture 274 formed therein for releasably engaging a barbed or angled arm 276 projecting from the distal end of the drive piston 278. Pressing the button 270 moves the release member 272 out of engagement with the arm 276 to release the piston 278 and fire the device. In the embodiment of FIG. 28, a trigger release member 280 is integraly molded with the drive piston 282.

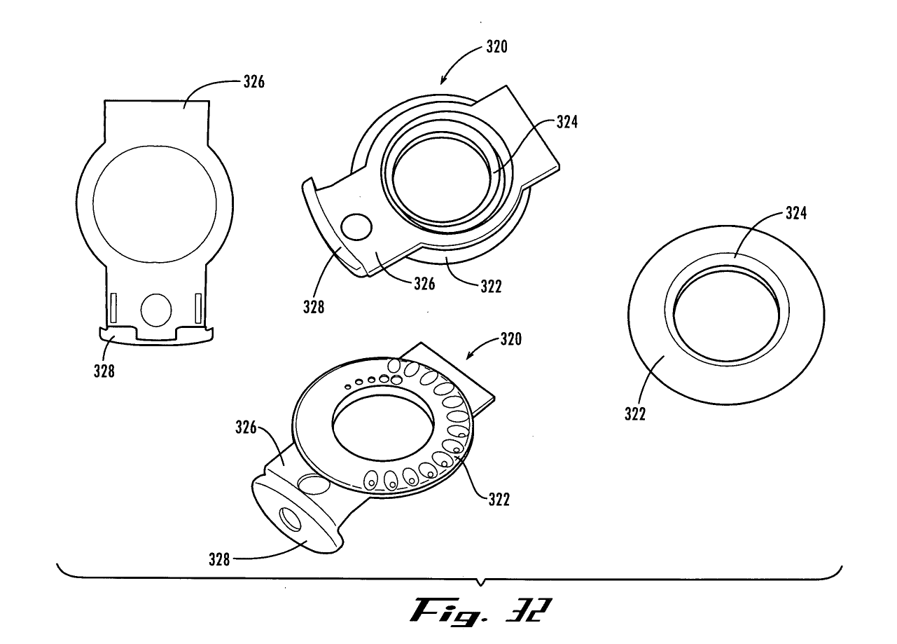

FIGS. 29a and 29b show a hinged trigger mechanism having a release button 290 on one side of a fulcrum 292, and a release or sear surface 294 on the opposite side of the fulcrum. Pressing the button 290 downward raises the sear surface 294 out of engagement with a cooperating trigger arm 296 projecting from the drive piston, releasing the drive piston to fire the device. A return spring 298 is preferably unitarily molded into the trigger mechanism.

In other embodiments, the device includes a trigger molded into the top cover of the lancet cartridge. For example, a trigger button and release arm may be integrally molded with the top cover, as by forming at least a portion thereof of a flexible material. In still other alternate embodiments, a cantilevered trigger arm extends alongside the drive piston, and has a free end in releasable engagement with a cooperating surface of the drive piston. The free end of the trigger arm is flexed out of engagement with the drive piston to fire the device.

Depth Adjustment

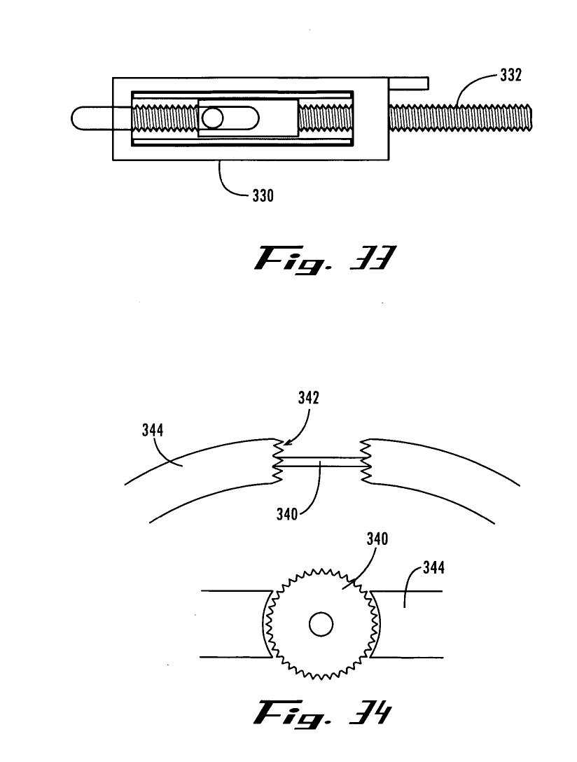

Example embodiments of the lancing device of the present invention preferably also include improved depth control features for enabling the user to selectively vary the depth of penetration of the lancet tip into the skin at the lancing site. For example, FIG. 30 shows a depth control mechanism according to an example embodiment of the invention, having a rotating plate 300 with multiple openings 302 therethrough for providing lancing depth adjustment. Depth adjustment can be accomplished, for example, by providing openings of different depth and/or diameter through a wall of the plate 300, by forming the wall of the plate to have different thicknesses at different points along its length, and/or by forming the wall to be radially offset by differing amounts at different angular positions. Rotation of the plate within the housing of the lancing device, for example by means of an external actuator member, brings the desired opening into alignment with the lancet opening through the housing.

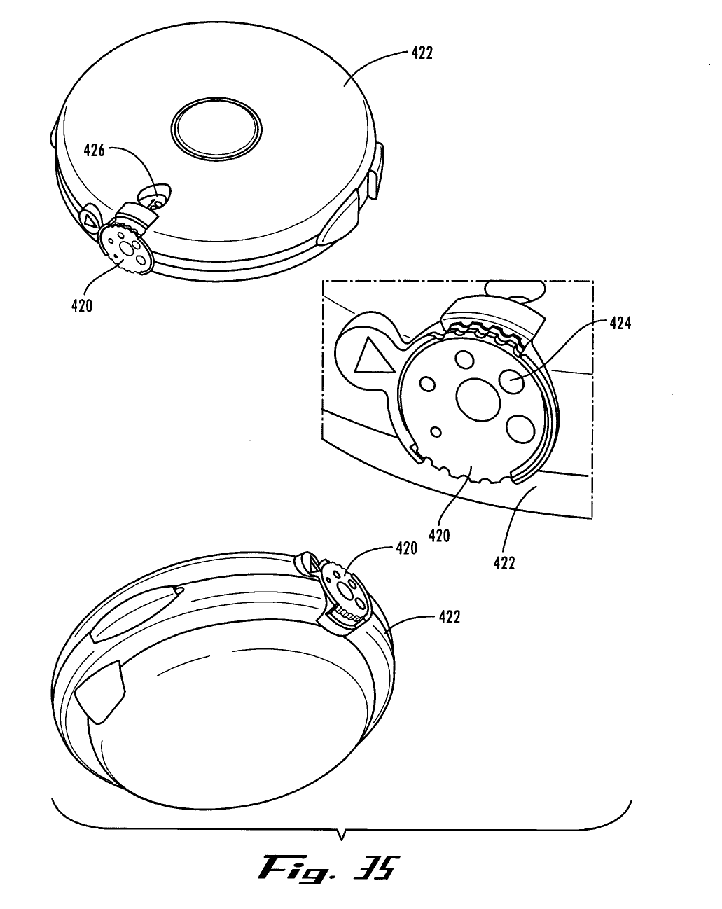

FIG. 31 shows a pivotal stroke-limiting depth control stop 310. The lancet impacts a generally arcuate contact face having a stepped surface with a plurality of stroke-limiting surfaces 312, each providing a different penetration depth. The user pivots the depth stop about an axis 314 to position the contact face with the selected portion of its stepped surface in line for contact with the active lancet to select the desired lancing depth.

FIG. 32 shows a sliding plate depth-control mechanism 320 with a dial member 322 having an eccentric hub 324 rotationally mounted within a cooperating opening of a translationally sliding plate member 326. Rotation of the dial 322 adjusts the position of a contact face 328 surrounding the lancet opening of the lancing device housing, to vary the depth of penetration.

In alternate embodiments, replaceable lancet cartridges are provided in different “sizes” for providing different lancing depths. For example, cartridges can be sold in “shallow”, “medium” and “deep” sizes, and the user purchases the desired size. The cartridges are interchangeable for use with a standard lancing device, and the variation in depth can be provided, for example, by varying the lancet needle length, the wall thickness, etc. In still other embodiments, a positional adjustment mechanism such as a screw-driven rack is provided for varying the position of the cartridge and/or the drive mechanism within the housing of the lancing device. In still other embodiments, the position or spring constant (stiffness) of the return spring and/or the drive spring of the lancet drive mechanism can be varied to provide depth control.

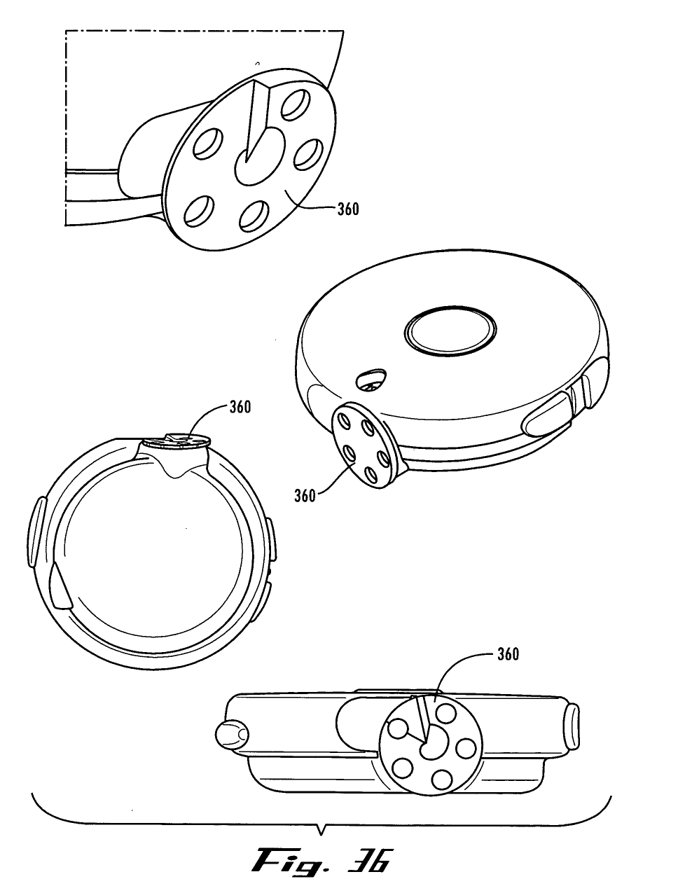

FIG. 33 shows a depth control mechanism incorporating a movable throttle plate 330 having front and back stops that move in tandem via actuation of a positioning screw 332 to provide depth adjustment.

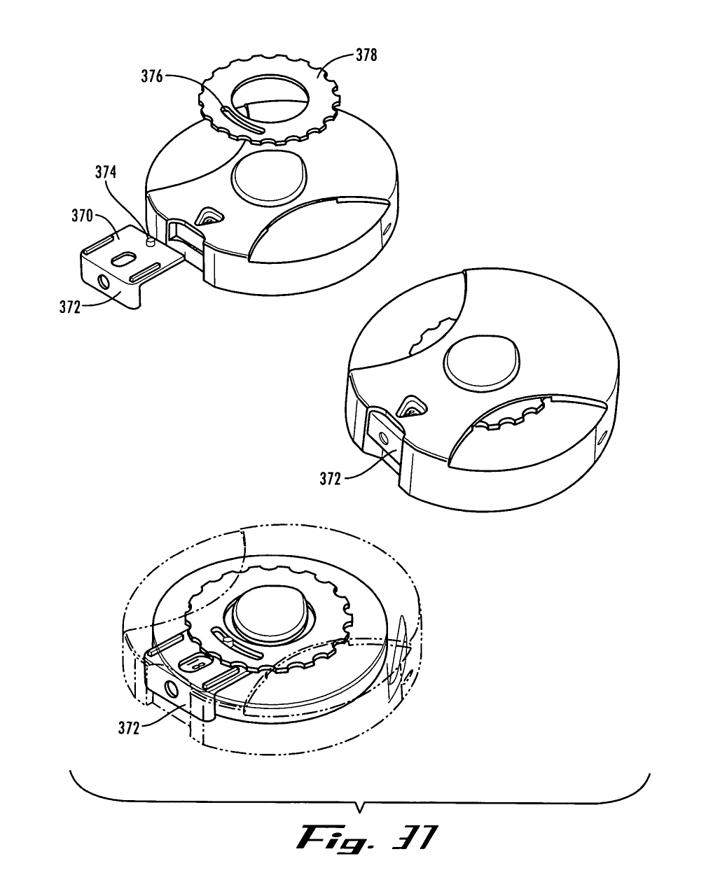

The depth control mechanism of FIG. 34 includes a circular threaded plate 340 with a lancet opening through its center, mounted within a cooperatively threaded opening 342 in the lancet device housing 344. The plate is turned to screw it in and out relative to the wall of the housing, providing a variable inner contact surface for limiting the stroke of the lancet, and/or a variable depth recess surrounding the lancet opening, for lancing depth control.

FIG. 35 shows a depth control mechanism comprising a circular depth wheel 420 rotationally mounted to the housing 422, and having a plurality of lancet openings 424 of differing diameter angularly offset from one another. The user rotates the wheel 420 to bring a selected one of the lancet openings into alignment with the path of travel of the active lancet, thereby varying the penetration depth. Numerical or other indicia 426 on or coupled to the depth wheel 420 are optionally provided, visible from the exterior of the housing, to inform the user of the selected penetration depth.

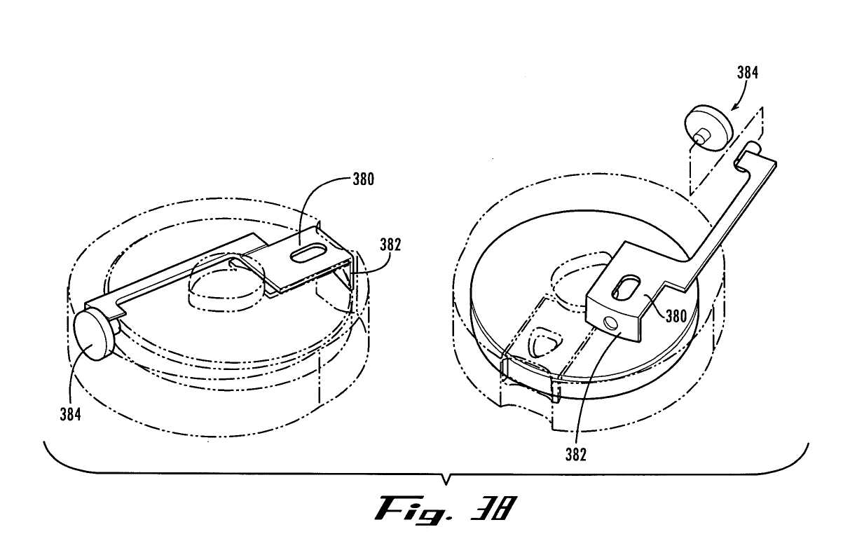

FIG. 36 shows a depth control mechanism comprising a tapered-thickness depth wheel 360 rotationally mounted to the housing. The depth wheel defines a plurality of angularly offset lancet openings, and has a wall thickness that varies about its circumference (i.e., different thicknesses at different angular displacements around the disk), The varying wall thickness allows the user to select the desired lancing depth by rotating the depth wheel to bring a selected one of the lancet openings into alignment with the path of travel of the active lancet.

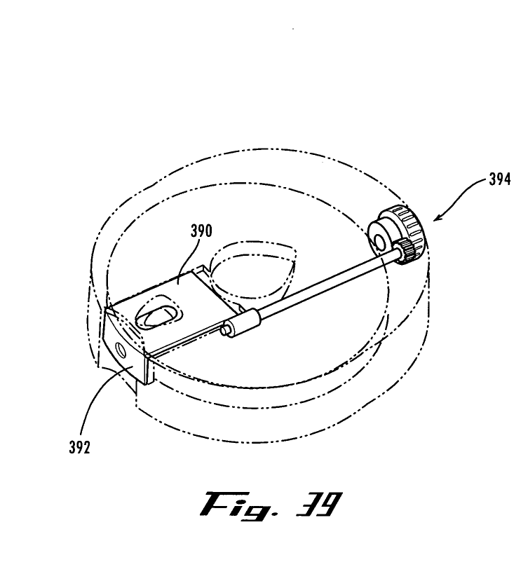

The depth control mechanism of FIG. 37 comprises a sliding plate 370 with an adjustable-position contact face 372 for placement against the lancing site to vary the depth of penetration. A pin 374 on the plate is engaged within an eccentricly arcuate cam slot 376 of a depth-adjustment wheel 378, which the user rotates to vary the position of the contact face relative to a lancet stroke-limiting surface of the housing, to adjust the penetration depth.

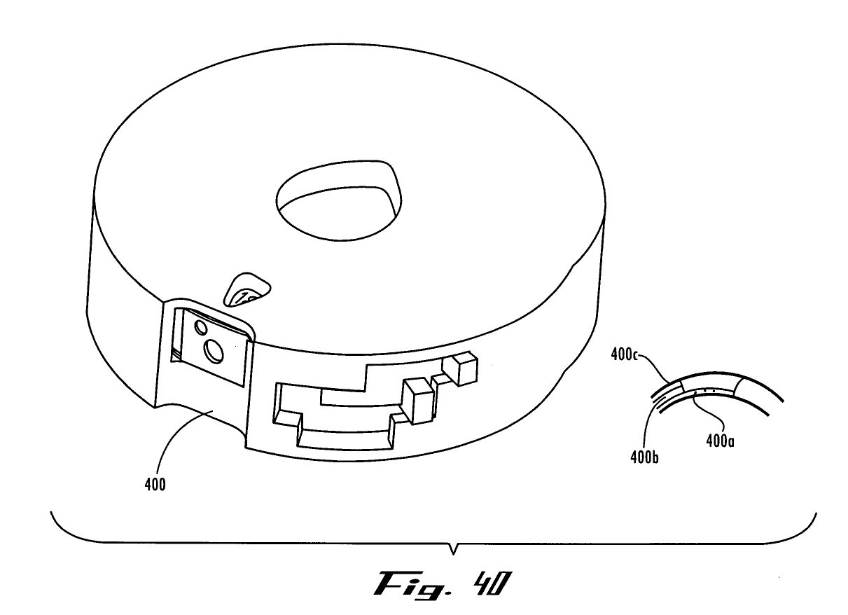

FIG. 38 shows a depth control mechanism having a sliding plate 380 with an adjustable-position contact face 382 for placement against the lancing site to vary the depth of penetration from lancing. A screw-drive mechanism 384 provides adjustment of the position of the contact face. FIG. 39 shows a similar depth control mechanism having a sliding plate 390 with an adjustable-position contact face 392 for placement against the lancing site to vary the depth of penetration from lancing. A gear-driven threaded rod 394, with an end engaged in a threaded sleeve portion of the sliding plate 390, provides adjustment of the position of the contact face.

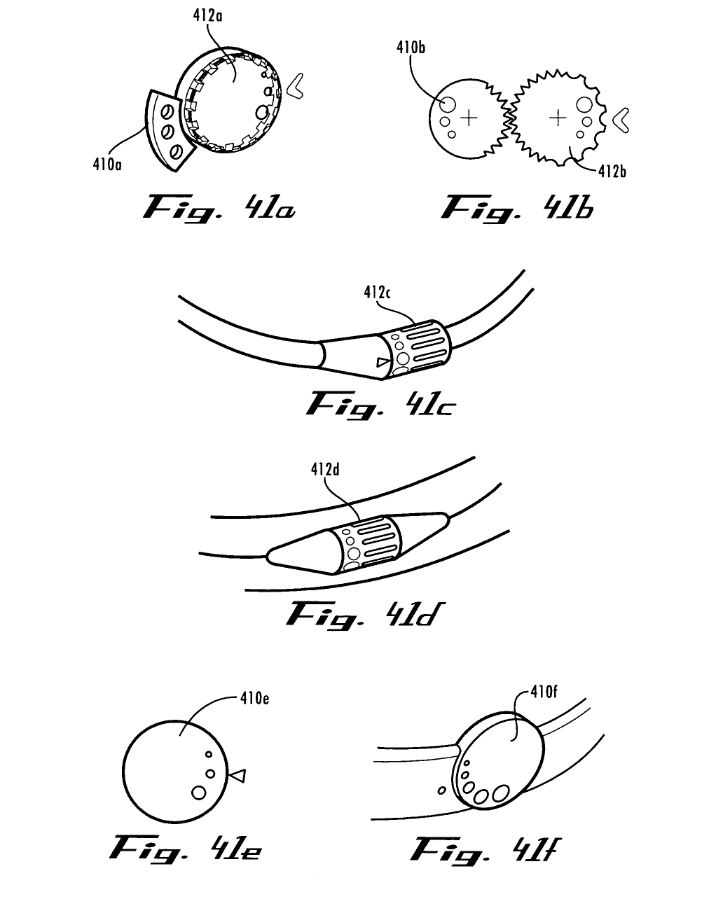

FIG. 40 shows a depth control mechanism having one or more shutters 400 for varying the effective wall thickness of the housing of the lancet device to control lancing depth. Each shutter has a lancet opening therethrough, and can be moved between a first position wherein its lancet opening is aligned with the path of travel of the active lancet, and a second position away from the path of travel of the active lancet. As successive shutters are moved to their respective first positions, their cumulative thickness increases the spacing between the forward face of the lancet body and the lancing site, thereby decreasing the penetration depth. Adjacent shutters are radially offset from one another, so that one, two, or more of the shutters can be selectively opened or closed to vary the lancing depth.

The depth control mechanism can comprise a depth control member 410 that is rigidly attached or integrally formed with an adjustment member 41, as shown for example in FIG. 41a, wherein an inclined depth control member having a varying thickness along its length projects radially from the adjustment knob. Alternatively, the depth control mechanism comprises separate depth control and adjustment members directly or indirectly coupled by gearing or other linkage means, as shown for example in FIGS. 41b-41f.

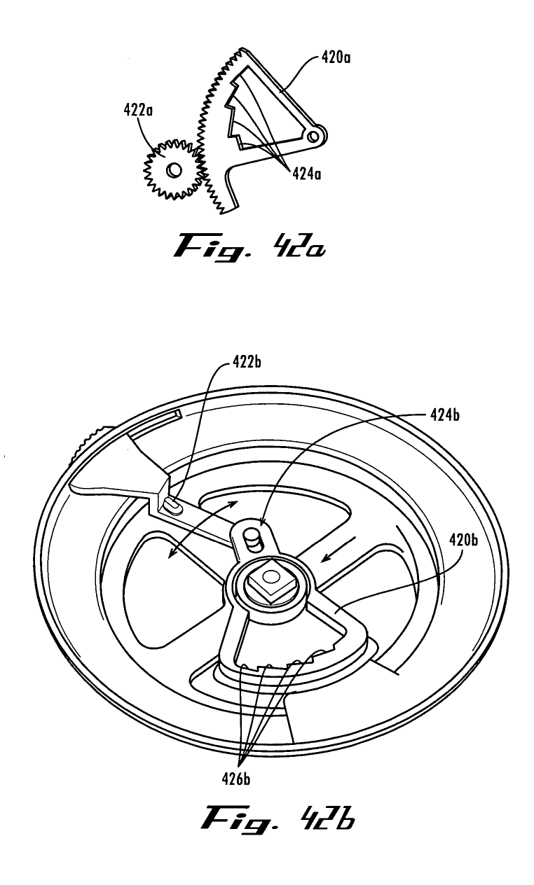

The angular position of the pivotal depth control member 420a of FIG. 42a is adjusted via gear drive 422a to selectively position one of the stepped contact faces 424a to limit the stroke of the lancet. The angular position of the pivotal depth control member 420b of FIG. 42b is adjusted via a toggle lingage 422b having a sliding pivot joint 424b, to selectively position one of the stepped contact faces 426b to limit the stroke of the lancet.

The depth control mechanism of FIG. 43 includes an axial spur gear 430 mounted to the adjustment knob 432, driving a toothed track 434 to align a selected contact face portion of the depth control member 436 to limit the stroke of the lancet and thereby control penetration depth. The depth control member can comprise a stepped contact face (436), or an inclined contact face (436′). A detent cantilever 438 is optionally provided, contacting the toothed surface of the spur gear 430 for indexing and tactile feedback to the user.

The depth control mechanism of FIG. 44 comprises an adjustable-width sliding iris having opposed halves 440a, 440b that can be moved closer to or further away from one another to reduce or increase the opening size, thereby varying the extent to which the subject's skin may bulge into the opening to vary penetration depth. A larger opening size allows the skin of the lancing site to be received further therein for deeper lancing, and a smaller opening size providing shallower lancing. FIG. 45 shows a depth control mechanism having a pivoting contact plate 450, the position of which is angularly variable relative to the position of the lancing cartridge 452, by means of a hinged connection 454 to the housing 456, to adjust the depth of penetration.

FIG. 46 shows a depth control mechanism having a contact face 460 for placement against the skin at the lancing site, the contact face comprising a tapered slot 462, wider at one end than at the other. Adjustment of the position of the tapered slot relative to the lancet opening 464 varies the effective opening size, thereby varying the depth of penetration.

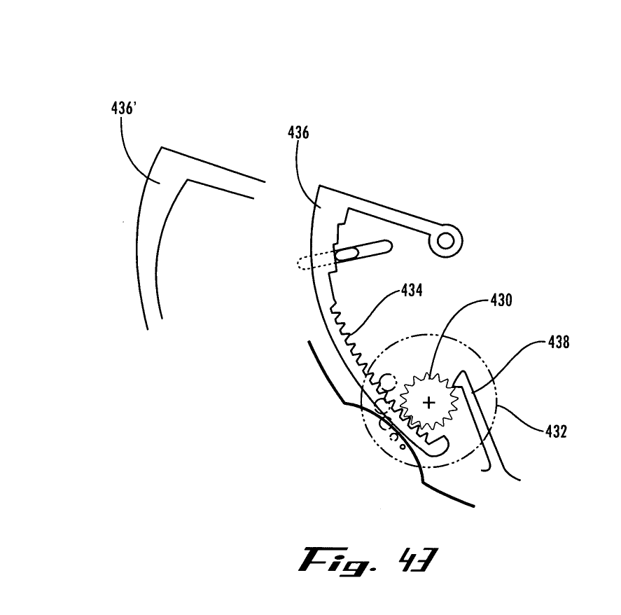

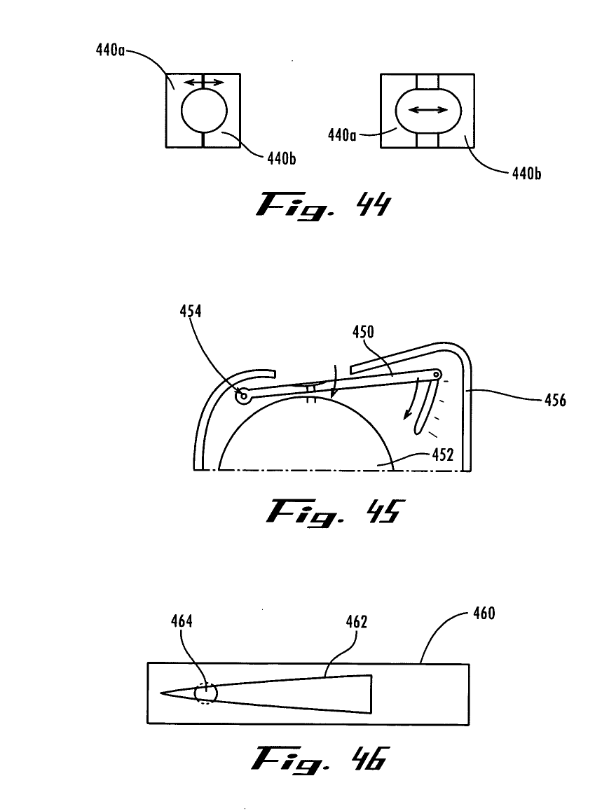

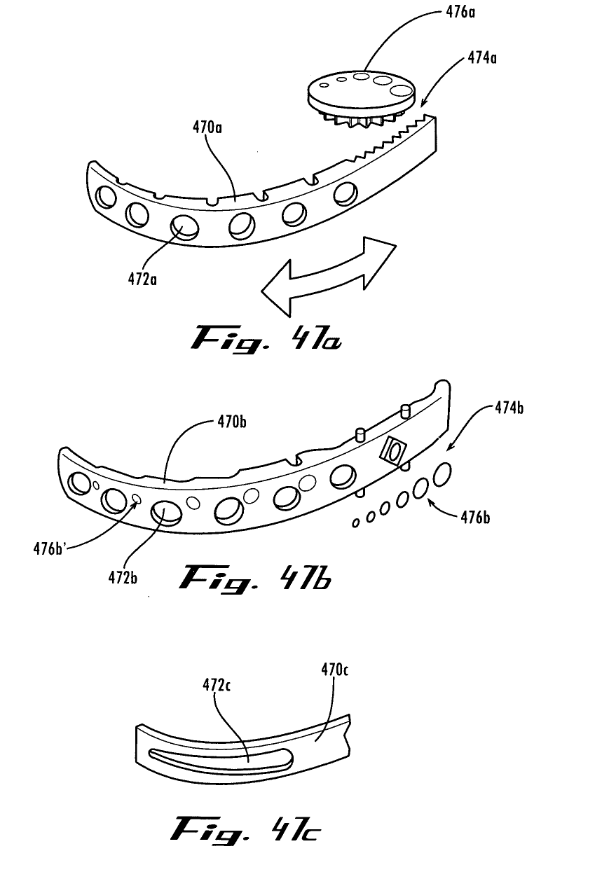

FIG. 47 shows a depth control mechanism having a flexible sliding band 470 defining a contact face for placement against the skin at the lancing site, and selectively movable across the housing in front of the housing's lancet opening. The thickness of the band 470 varies along its length, and/or the dimension of the opening(s) 472 through the band varies, to provide lancing depth adjustment. A geared adjustment knob 474a, or pin-and-detent slide coupling 474b provide positional adjustment of the band 470 relative to the housing. The geared face of the adjustment knob 474a can operate vertically on a toothed surface on the front or back face of the band as shown, or can operate horizontally on a toothed surface on the top or bottom edge of the band. Depth indicators can be provided on or adjacent the adjustment knob (476a) or the slide coupling (476b); and/or along the band (476b′) for viewing through a window adjacent the openings. Optionally, the band 470 can be moved completely out of the way of the lancet opening through the housing, to provide maximum lancing depth, as for alternate site lancing. In alternate forms, the band slides within the housing, behind a contact surface for placement against the skin at the lancing site, to limit the stroke of the lancet and/or to vary the opening size, and thereby control penetration depth.

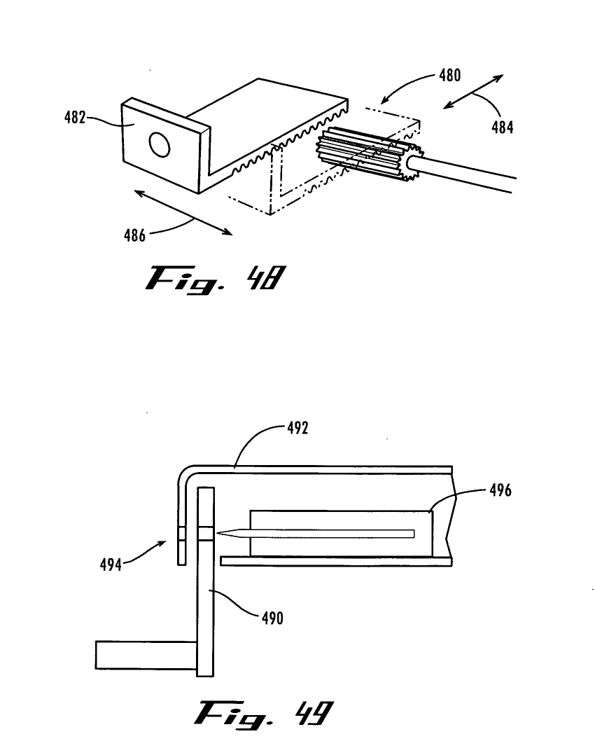

The depth control mechanism of FIG. 48 comprises a rack-and-pinion drive mechanism 480 for advancing and retracting the position of a sliding plate 482 along a first axis (indicated by directional arrow 484), the sliding plate having a contact face for contact with the skin at the lancing site, to adjust lancing depth. Optionally, the sliding plate 482 can slide along a second axis (indicated by directional arrow 486), perpendicular to the first axis, to move the plate out of the path of the active lancet or maximum penetration depth, as for alternate site lancing.

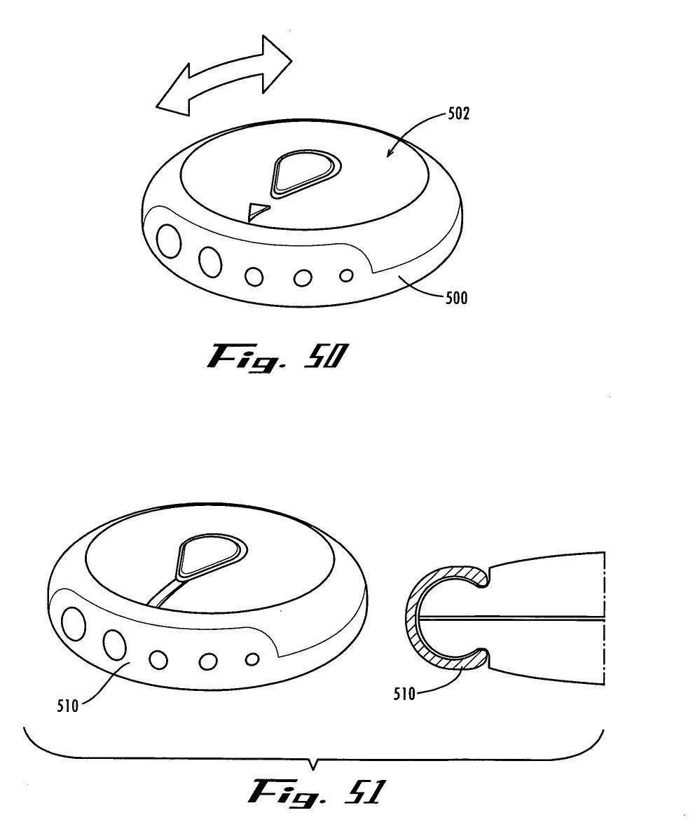

FIG. 49 shows a depth control mechanism having a depth adjustment insert member 490 with a flange selectively insertable or removable to and from the device housing 492 adjacent the lancet opening 494, to limit the stroke of the lancet 496 and thereby vary depth of penetration.

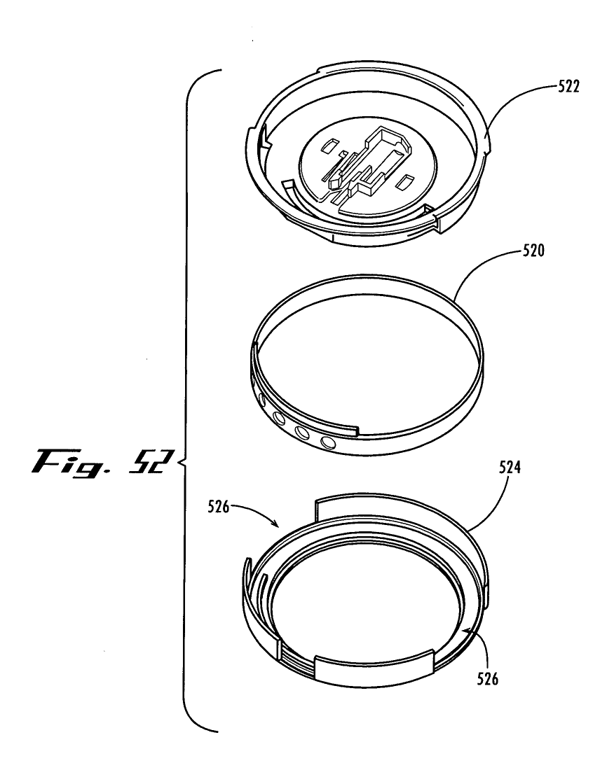

FIG. 50 shows a depth control mechanism having an outer housing 500 with openings of different sizes, and an inner mechanism 502 rotationally mounted within the housing to select one of the openings for alignment with the active lancet, and thereby control lancing depth. FIG. 51 shows a depth control mechanism having an external sliding plate 510 with varying wall thickness and/or opening sizes, which is selectively movable along the housing to align one of the openings with the active lancet and thereby vary the lancing depth. The sliding plate 510 optionally serves also as a latch to secure the upper and lower housing halves to one another.

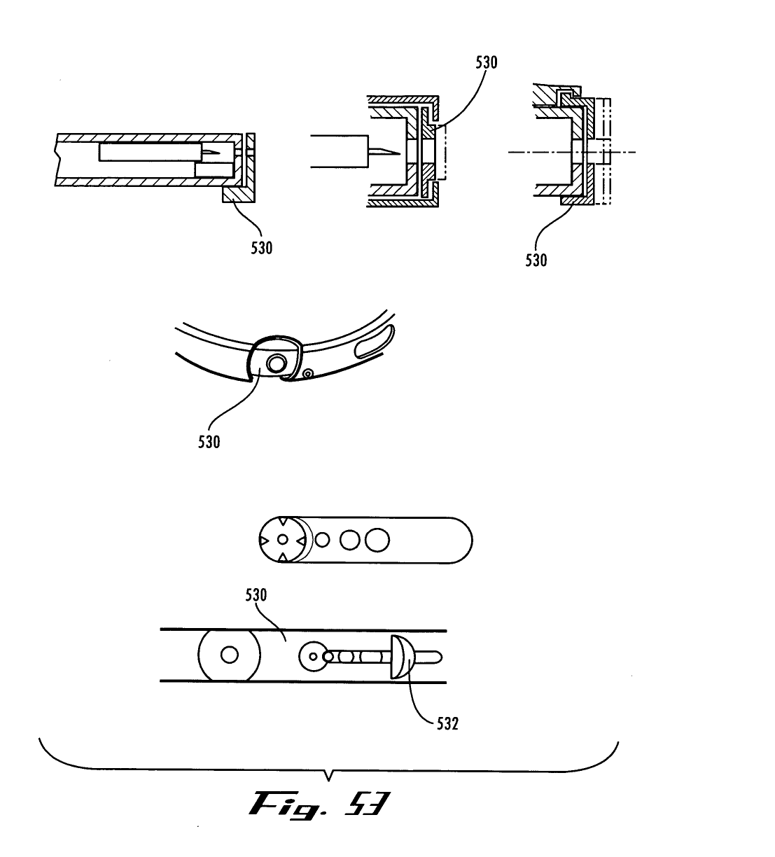

FIG. 52 shows a depth control mechanism having a continuous depth adjustment ring 520 rotationally captured between the lancing device mechanism base 522 and the bottom housing enclosure 524. The ring 520 has varying opening sizes and/or wall thickness to control lancing depth. One or more portions of the ring 520 are accessible from the exterior of the housing through opening(s) 526 in the bottom housing enclosure 524, to permit rotational adjustment of the ring by the user.

FIG. 53 shows a depth control mechanism having a depth adjustment band 530 movably mounted along the outside of the housing, and having varying opening sizes and/or wall thickness to control lancing depth. A sliding adjustment knob 532 is preferably provided for controlling the positioning of the depth adjustment band 530.

While the invention has been described with reference to preferred and example embodiments, it will be understood by those skilled in the art that a variety of modifications, additions and deletions are within the scope of the invention, as defined by the following claims.

1. A lancing device comprising a plurality of lancets; an advancing mechanism for sequentially advancing the lancets in a first direction and bringing an active one of the lancets into engagement with a drive mechanism; and an anti-reverse mechanism for preventing advancement of the lancets in a second direction opposite the first direction.

2. The lancing device of claim 1, wherein the plurality of lancets are arrayed within a multi-lancet cartridge.

3. The lancing device of claim 1, wherein the anti-reverse mechanism comprises a ratchet and pawl.

4. The lancing device of claim 1, wherein the anti-reverse mechanism comprises a torsion spring.

5. The lancing device of claim 1, wherein the anti-reverse mechanism comprises a spring-loaded plunger.

6. The lancing device of claim 1, wherein the advancing mechanism comprises a rotationally-mounted advancing knob.

7. The lancing device of claim 6, wherein the advancing knob is rotational in a first rotational direction only.

8. The lancing device of claim 6, wherein the advancing knob is rotational in a first rotational direction along an advancing stroke to advance the lancets, and in a second rotational direction opposite the first rotational direction along a return stroke to return the advancing mechanism to a ready state.

9. The lancing device of claim 8, further comprising means for preventing partial advancement of the lancets by preventing return of the advancing mechanism in the second direction until a full advancing stroke is completed.

10. A lancing device comprising a replaceable multi-lancet cartridge, a drive mechanism for driving an active lancet of the multi-lancet cartridge between a cocked position and a lancing position, and an interlock for preventing advancement of the cartridge when the drive mechanism is cocked.

11. The lancing device of claim 10, wherein the interlock comprises an arm projecting from the drive mechanism, for engaging a cooperating surface feature of the multi-lancet cartridge when the drive mechanism is cocked.

12. The lancing device of claim 10, wherein the interlock comprises a locking bolt that is retracted upon contact with the drive mechanism when cocked, to engage the lancet cartridge and lock it in position.

13. A lancing device comprising a plurality of lancets in an array, and an advancing mechanism to advance through the array of lancets and successively bring each of the plurality of lancets into an active position, wherein the advancing mechanism comprises indexing means to prevent partial advancement of the array to a position where no lancet is in the active position.

14. The lancing device of claim 13, wherein the array of lancets are in a replaceable multi-lancet cartridge.

15. The lancing device of claim 14, wherein the indexing means comprises a series of spaced recesses formed in the exterior of the multi-lancet cartridge, and a flexible member for releasable engagement within at least one of the recesses.

16. The lancing device of claim 15, wherein the flexible member comprises an arm having a first angled finger for engagement with one of the recesses of the lancet cartridge, and a second angled finger for tracking a cam surface to flex the first angled finger into and out of engagement with the cartridge.

17. A lancing device comprising a replaceable multi-lancet cartridge having a plurality of lancets associated therewith, and lancet retaining means to prevent the lancets from being displaced from the cartridge until advanced into an active position.

18. The lancing device of claim 17, wherein the lancet retaining means comprise a split retaining ring.

19. The lancing device of claim 17, wherein the lancet retaining means comprise a plurality of molded cantilevers formed in a top cover portion of the cartridge.

20. The lancing device of claim 17, wherein the lancet retaining means comprises at least one resilient hold-down snap for retaining each lancet in the cartridge.

21. The lancing device of claim 17, wherein the retaining means further comprise a limit member to prevent re-use of lancets in the cartridge.

22. A replaceable multi-lancet cartridge for a lancing device, the cartridge comprising a plurality of lancets, each lancet having a removable endcap associated therewith, and at least one spring for biasing removed endcaps out of a path of travel of their associated lancets, said at least one spring being initially flat, and being flexed upon assembly into the cartridge.

23. A lancing device comprising a drive piston, a drive spring for advancing the drive piston, and a return spring for retracting the drive piston, wherein one of the drive spring and the return spring surrounds an exterior portion of the drive piston, and the other of the drive spring and the return spring is captured within an internal recess of the drive piston.

24. A lancing device comprising a drive piston movable between a cocked position and a fired position, the lancing device further comprising a trigger mechanism having a release member defining an aperture, wherein an expanded portion of the drive piston is releasably engageable within the aperture of the release member to retain the drive piston in its cocked position until released.

25. A lancing device for use in combination with a multi-lancet cartridge comprising a plurality of lancets, the lancing device comprising a housing, a drive mechanism, an advancing mechanism for bringing the lancets of the multi-lancet cartridge sequentially into engagement with the drive mechanism, and a depth-control mechanism providing adjustment of a depth of penetration of an active one of the lancets.
